(19) United States
(12) Patent Application Publication
(10) Pub. No.: US 2007/0218498 A1
(43) Pub. Date: Sep. 20, 2007

(54) Use of soluble FLT-1 and its fragments in cardiovascular conditions

(76) Inventors: Kenneth F. Buechler, Rancho Santa Fe, CA (US); Paul H. McPherson, Encinitas, CA (US)

Correspondence Address: Richard J. Warburg;FOLEY & LARDNER LLP, P.O. Box 80278, San Diego, CA, 92138-0278, US

(21) Appl. No.: 11/512,743

(22) Filed: Aug. 29, 2006

Related U.S. Application Data
(60) Provisional application No. 60/712,599, filed on Aug. 30, 2005.

(51) Int. Cl. G01N 33/53

(52) U.S. Cl. 435/7.1

(57) ABSTRACT

The present invention relates to materials and procedures for evaluating patients suffering from cardiovascular conditions, particularly acute coronary syndromes. In particular, an assay configured to measure the level of soluble FLT-1 in a patient sample, alone or in combination with one or more other markers, provides diagnostic and/or prognostic information. While applicable to diseases and conditions in which inflammation is generally manifested, the methods and compositions described herein are particularly applicable to acute coronary syndromes, including conditions selected from the group consisting of stable angina, unstable angina, non-ST-elevation non-Q wave myocardial infarction, ST-elevation non-Q wave MI, and transmural (Q-wave) MI.

### FIELD OF THE INVENTION

The present invention relates in part to methods, compositions, and devices for the measurement of soluble FLT-1 and/or its fragments, and the use of such measurement in the diagnosis, prognosis, and treatment of patients with cardiovascular conditions.

### BACKGROUND OF THE INVENTION

The following discussion of the background of the invention is merely provided to aid the reader in understanding the invention and is not admitted to describe or constitute prior art to the present invention.

The term “cardiovascular conditions” refers to a diverse set of disorders of the heart and vasculature, including atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, peripheral vascular disease, and acute coronary syndromes. Major cardiovascular conditions may present with few overt symptoms, such as pain, dyspnea, weakness, palpitations, and dizziness. The clinical presentation of these various conditions can often be strikingly similar, even though the underlying disease, and the appropriate treatments to be given to one suffering from the various diseases, can be completely distinct.

Workers seeking to provide rapid diagnostic (that is, the presence of a particular condition or disease) and/or prognostic (that is, a prediction of some future outcome) information for various cardiovascular diseases or conditions have sought to identify subject-derived “markers” that are indicative of a particular diagnosis or prognosis. In the case of a “diagnostic marker,” these are molecules that are preferably present in a sample obtained from a first subject suffering from a condition or disease in an amount that differs (either a greater or lesser amount) from the amount present in a sample from a second “normal” subject. In the case of a “prognostic marker,” these are molecules that are preferably present in a sample obtained from a first subject predisposed to some future outcome in an amount that differs from the amount present in a sample from a second subject (e.g., a subject suffering from the same condition as the first subject, a subject suffering from a different condition, or a normal subject).

For example, epidemiological studies have shown an association between circulating levels of certain inflammatory markers and coronary artery disease (CAD). C-reactive protein (“CRP”) has been reported to be predictive for future coronary events. See, e.g., Kuller et al., Am. J. Epidemiol. 144:537-47, 1996; Haverkate et al., Lancet 349:462-66, 1997; Ridker et al., N. Engl. J. Med. 342:836-43, 2000. Other markers, including cardiac-specific troponin, CK-MB, myoglobin, interleukin (“IL”)-6, soluble adhesion molecules, IL-18, and tumor necrosis factor-α (“TNF-α”) have also been reported to be potential tools for cardiovascular diagnosis and/or risk prediction. See, e.g., Ridker et al., Circulation 101:1767-72, 2000; Volpato et al., Circulation 103:947-53, 2001; Ridker et al., Lancet 351:88-92, 1998; Ridker Circulation 103:491-95, 2001; Barbaux et al., Arterioscler. Thromb. Vasc. Biol. 21:1668-73, 2001; Blankenberg et al., Circulation 104:1336-42, 2001; Blankenberg et al., Circulation 106:24-30, 2002; Koukkunen et al., Ann. Med. 33:37-47, 2001; Ridker et al., Circulation 101:2149-53, 2000; Rallidis et al., Heart 90:25-9, 2004.

FLT-1, also known as vascular endothelial growth factor receptor 1 (Swiss Prot P17948) is the receptor for VEGF, VEGFB, and placental growth factor. It is a type 1 membrane protein with an N-terminal extracellular domain connected via a transmembrane domain to a C-terminal cytoplasmic domain. A soluble FLT-1 splice variant (sFLT-1, Swiss Prot P17948-1) exists, together with possible cleaved soluble extracellular portions of the parent membrane protein. See, e.g., U.S. Pat. No. 5,712,380. It has been reported that levels of sFLT-1 are lower in acute and chronic myocardial infarction as compared to control subjects.

There remains in the art the need to identify markers useful in evaluating patient diagnosis and prognosis within the spectrum of cardiovascular conditions, so that patients at risk of morbidity and/or death or can be identified and treated.

### SUMMARY OF THE INVENTION

The present invention relates to materials and procedures for diagnosing subjects suffering from one or more cardiovascular conditions or diseases, and/or for evaluating the prognosis of such subjects. The materials and procedures described herein can be used to identify those individuals suffering from cardiovascular conditions, and/or that may be at increased risk for one or more serious complications, including the risk of death, resulting from one or more cardiovascular conditions, each of which may be used to guide the clinician in treatment of such individuals.

In a first aspect of the invention, sFLT-1, and/or its fragments, are used to provide diagnostic and/or prognostic information on a subject. In this approach, a sample obtained from a subject is measured using an assay configured to detect sFLT-1. Such assays may be specific for sFLT-1, may bind one or more fragments of the protein in addition to sFLT-1, and/or may be specific for one or more fragments of sFLT-1 in that they do not also bind intact sFLT-1. In preferred embodiments, an increase in the measured protein, relative to a protein level in normal subjects, is indicative of the presence of a cardiovascular condition in the subject from whom the sample is obtained.

In preferred embodiments, the results obtained from an sFLT-1 assay of the present invention may be related to the presence or absence of one or more conditions in the subject within the scope of acute coronary syndrome, preferably selected from the group consisting of acute myocardial infarction (AMI), acute ST elevation myocardial infarction (STEMI), acute non-ST elevation myocardial infarction (NSTEMI), unstable angina (UA), and stable angina (SA). Such assays may also be used in the diagnosis of acute myocardial infarction within 0-3 hours of the event (the onset of myocardial infarction), acute myocardial infarction within 0-6 hours of the event, non-ST elevation myocardial infarction within 0-3 hours of the event, non-ST elevation myocardial infarction within 0-6 hours of the event, ST elevation myocardial infarction within 0-3 hours of the event, ST elevation myocardial infarction within 0-6 hours of the event, and troponin I-negative (TNI−) non-ST elevation myocardial infarction, ST elevation myocardial infarction, unstable angina, and stable angina sFLT-1 assays of the present invention are configured such that a change in the signal obtained from the assays, relative to a signal indicative of normal subjects, is indicative of the presence of a cardiovascular condition and/or of a particular prognosis. The level of a particular marker in a subject population will be represented by a distribution of values, and in “disease” and “normal” populations, the distribution will typically contain some overlap. In such a case, there is no absolute threshold that separates the two populations. Rather, there are numerous possible thresholds that can be selected, with the selection of any particular threshold involving a consideration of specificity and sensitivity for the assay. Methods for determining a level indicative of “normal” and “diseased” subjects, such as Receiver Operating Characteristic (ROC) analysis, are well known to those of skill in the art. See, e.g., Zweig and Campbell, Clin. Chem. 39: 561-77 (1993).

In accordance with the foregoing, particularly preferred methods comprise performing an assay configured to detect soluble FLT-1 on a sample obtained from a subject, and performing one or more of the following determinations: diagnosing the presence of a cardiovascular condition if the assay result is greater than a predetermined threshold soluble FLT-1 level; or diagnosing the absence of a cardiovascular condition if the assay result is less than a predetermined threshold soluble FLT-1 level; or assigning an increased likelihood of a poor prognostic outcome if the assay result is greater than a predetermined threshold soluble FLT-1 level, relative to a prognostic risk assigned if the assay result is less than the threshold soluble FLT-1 level; or assigning a decreased likelihood of a poor prognostic outcome if the assay result is less than a predetermined threshold soluble FLT-1 level, relative to a prognostic risk assigned if the assay result is greater than the threshold soluble FLT-1 level. This is not meant to indicate that the diagnosis or prognosis is necessarily made solely on the basis of the soluble FLT-1 assay result alone, as correlating a marker measurement to a diagnosis or prognosis could also combine an assay result with other assay results, with clinical indicia (e.g., an electrocardiogram result, etc.

Preferred antibodies for use in the sFLT-1 assays of the present invention are described hereinafter in terms of the DNA and protein sequences encoding the heavy and light chain variable regions of the preferred antibodies. These sequences and the proteins encoded thereby can be used in antibody engineering methods (such as CDR grafting, chain shuffling, and mutagenesis) to derive new antibodies that bind to the same or related epitopes as those bound by the preferred antibodies. Alternatively, or in addition, the preferred antibodies may be used to screen monoclonal or polyclonal antibodies and antibody libraries (e.g., phage display libraries) to identify alternative antibodies that bind to the same or related epitopes as those bound by the preferred antibodies.

While the sFLT-1 assays described herein may be used alone to provide diagnostic and/or prognostic information, in various embodiments such sFLT-1 assays are used in combination with one or more additional subject-derived marker assays to provide diagnostic and/or prognostic information. Assays configured to detect one or more such additional markers can preferably be combined with the sFLT-1 assays described herein to increase the predictive value test results as a diagnostic or prognostic indicator. The phrase “increases the predictive value” thus refers to the ability of two or more combined markers to improve the ability to provide a diagnosis or a prognosis, in comparison to a prediction obtained from sFLT-1 reults alone.

In various embodiments, one or more additional markers are independently selected from the group consisting of markers related to myocardial injury, markers related to apoptosis, markers related to blood pressure regulation, markers related to inflammation, and markers related to coagulation and inflammation. Preferred additional markers of the invention are B-type natriuretic peptide (BNP), proBNP, NT-proBNP, BNP₃₋₁₀₈, one or more cardiac-specific troponins (e.g., cardiac troponin I and/or T), caspase-3, C-reactive protein, creatine kinase-MB (CKMB), fibrinogen, IL-6, IL-8, IL-18, MMP-9, heart-type fatty acid binding protein, monocyte chemoattractant protein-1 (MCP-1), myeloperoxidase (MPO), myoglobin, NT-proBNP, thrombus precursor protein (TpP), TNF-α, D-dimer, sCD40L, and/or markers related thereto. This list is not meant to be limiting, and additional subject derived markers for use are described hereinafter. In addition, non-subject-derived markers such as ST-segment depression, age, smoking status, diabetes, ejection fraction, hypertension, and/or prior MI may also be used as additional variables that may be combined with an sFLT-1 assay results in a subject sample.

The skilled artisan will understand that the plurality of markers need not be determined in the same sample, or even at the same time. For example, one marker may be an early marker of ACS, while another may not appear in serum samples from the same subject until some time has passed from the onset of ACS.

The phrase “determining the diagnosis” as used herein refers to methods by which the skilled artisan can determine the presence or absence of a particular disease or condition in a patient. The term “diagnosis” does not refer to the ability to determine the presence or absence of a particular disease or condition with 100% accuracy, or that a given course or outcome is more likely to occur than not. Similarly, the phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given characteristic, such as the presence or level of a prognostic indicator, when compared to those individuals not exhibiting the characteristic.

ROC curve analysis may be employed to assign a threshold value above which (or below which, depending on how a marker changes with the disease) the test is considered to be indicative of one state or condition (e.g., presence of disease, assignment to a prognosis group) and below which the test is considered to be indicative of another state or condition (e.g., absence of disease, or assignment to another prognosis group). In certain embodiments, an sFAS_{Δint} threshold level may be selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

The term “correlating,” as used herein in reference to the use of one or more diagnostic and/or prognostic indicator(s), refers to comparing the presence or amount of the diagnostic indicator in a subject sample to its presence or amount in subjects known to suffer from, or known to be at risk of, a given condition; or in subjects known to be free of a given condition. In certain embodiments, sFLT-1 assay results may be correlated to a diagnosis or prognosis by merely the presence or absence of the polypeptide(s) being measured in the sFLT-1 assay. For example, an assay can be designed so that a positive signal for a marker only occurs above a particular threshold concentration of interest, and below which concentration the assay provides no signal above background. In other embodiments, threshold concentration(s) of sFLT-1 can be established, and the level of sFLT-1 in a patient sample can simply be compared to the threshold level(s). In addition, numerous multivariate methods for assigning a diagnosis and/or prognosis on the basis of multiple markers are well known in the art. Preferred methods for correlating multiple markers to a diagnosis and/or prognosis are described hereinafter.

The skilled artisan will understand that associating one or more diagnostic or prognostic indicators with a disease or a predisposition to a particular outcome is a statistical analysis. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Often, a positive likelihood ratio, negative likelihood ratio, odds ratio, or hazard ratio is used as a measure of a test's ability to predict risk or diagnose a condition or disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, the assays of the present invention may be preferably configured to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, the assays of the present invention may be preferably configured to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, the assays of the present invention may be preferably configured to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

In certain preferred embodiments, the methods of the present invention are applied to diagnose a subject as suffering from an acute coronary syndrome, and/or to assign a prognosis to a subject so diagnosed. The phrase “acute coronary syndromes” as used herein refers to a group of coronary disorders that result from ischemic and/or necrotic insult to the heart. ACS includes unstable angina, non-ST-elevation non-Q wave MI, ST-elevation non-Q wave MI, and transmural (Q-wave) MI. ACS can be divided into non-ST-elevation ACS and ST-elevation ACS, each of which may be associated with certain prognostic indicators and prognoses, as described herein. The phrase “non-ST-elevation acute coronary syndrome” refers to those ACS not associated with an elevated ST component in an electrocardiogram. Non-ST-elevation ACS include unstable angina and non-ST-elevation non-Q wave MI. See, e.g., Nyman et al., J. Intern. Med. 1993; 234: 293-301, 1993; Patel et al., Heart 75: 222-28, 1996; Patel et al., Eur. Heart J. 19: 240-49, 1998; and Lloyd-Jones et al., Am. J. Cardiol. 81: 1182-86, 1998.

Diagnosis of ACS generally, and non-ST-elevation ACS in particular, is well known to the skilled artisan. See, e.g., Braunwald et al., Unstable angina: diagnosis and management, Clinical practice guideline no. 10 (amended), AHCPR publication no. 94-0602. Rockville, Md.: Department of Health and Human Services, 1994; Yusuf et al., Lancet 352:507-514, 1998; Savonitto et al., JAMA 281:707-713, 1999; Klootwijk and Hamm, Lancet 353 (suppl II): 10-15, 1999.

In another aspect, the invention relates to methods for determining a diagnostic and/or prognostic panel comprising a plurality of prognostic markers, one of which is an sFLT-1 assay results, that can be used to assign a diagnosis of a cardiac condition, preferably an acute coronary syndrome, and or to assign a prognosis to a patient diagnosed with a cardiovascular condition. Once the plurality of markers has been determined, the levels of the various markers making up the panel can be measured in one or more patient sample(s), and then compared to the diagnostic levels determined for each marker, as described above.

It is yet another object of the invention to provide methods for determining and/or monitoring a treatment regimen for use in a patient diagnosed with a cardiovascular disease, most preferably an acute coronary syndrome. The methods preferably comprise determining an sFLT-1 assay results using an assay configured as described herein. One or more treatment regimens appropriate for the particular prognosis and/or diagnosis can then be used to treat the patient. With regard to monitoring a course of treatment, changes in the one or more markers measured may be used to assess changes in the patient's health status resulting from a treatment regimen.

It is yet another object of the invention to provide kits for determining the prognosis and/or diagnosis of a patient diagnosed with a cardiovascular disease, most preferably an acute coronary syndrome. These kits preferably comprise devices and reagents for performing an sFLT-1 assay as described herein, and instructions for performing the assay. Optionally, the kits may contain one or more methods, such as a threshold value to be used for comparison of a measured value, for converting an sFLT-1 assay result to a prognosis or diagnosis. Additionally, the kits may provide devices and reagents for determining one or more additional prognostic markers to be combined with an sFLT-1 assay result in a patient sample.

In preferred embodiments, such kits preferably comprise at least one antibody, and most preferably two or more antibodies, selected from amongst the preferred antibodies described hereinafter, and/or one antibody, and most preferably two or more antibodies, that bind to the same epitope or a related epitope to those bound by one or more of the preferred antibodies described hereinafter.

### BRIEF DESCRIPTION OF THE FIGURES

FIG. 1 depicts the heavy chain variable region (SEQ ID NO:1) and light (kappa) chain variable region amino acid sequence (SEQ ID NO:2) obtained from a preferred antibody of the present invention designated CA0071 Z2ZM 01941.

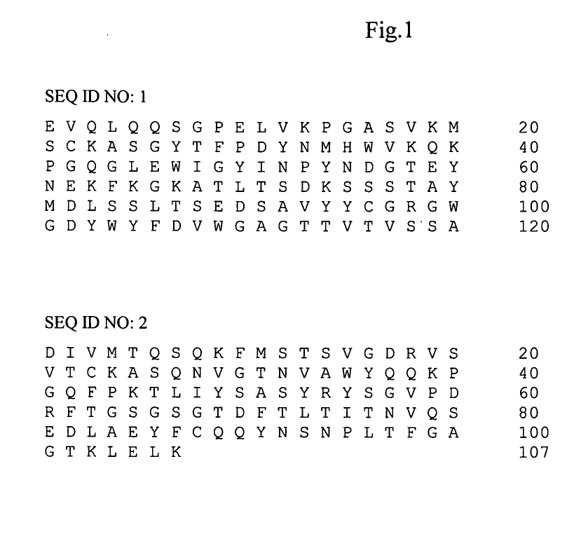

FIG. 2 depicts the heavy chain variable region (SEQ ID NO:3) and light (kappa) chain variable region amino acid sequence (SEQ ID NO:4) obtained from a preferred antibody of the present invention designated CA0071 Z2ZA 01171.

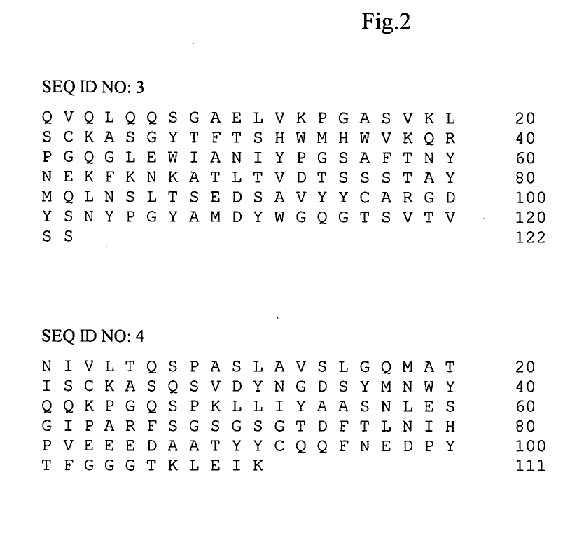

FIG. 3 depicts the heavy chain variable region (SEQ ID NO:5) and light (kappa) chain variable region amino acid sequence (SEQ ID NO:6) obtained from a preferred antibody of the present invention designated CA0071 Z2ZB 01171.

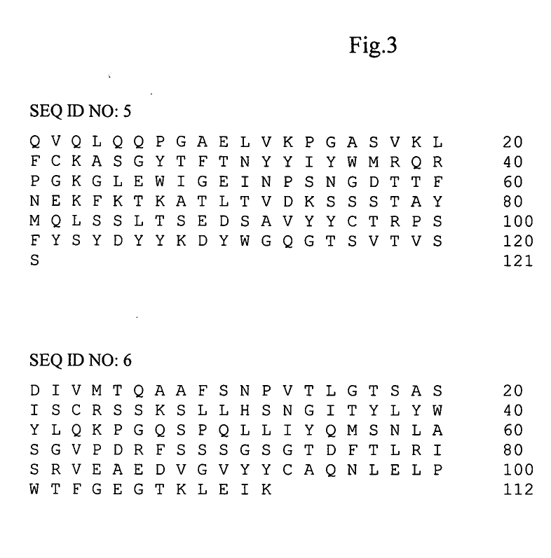

FIG. 4 depicts the heavy chain variable region (SEQ ID NO:7) and light (kappa) chain variable region nucleic acid sequence, together with the translated amino acid sequence, (SEQ ID NO:8) obtained from a preferred antibody of the present invention designated CA0071 Z2ZM 01941.

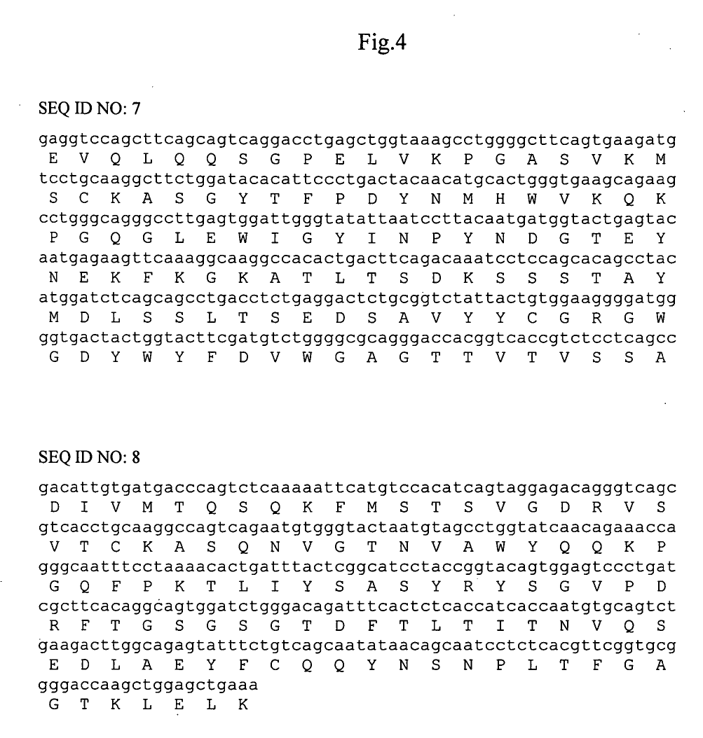

FIG. 5 depicts the heavy chain variable region (SEQ ID NO:9) and light (kappa) chain variable region nucleic acid sequence, together with the translated amino acid sequence, (SEQ ID NO:10) obtained from a preferred antibody of the present invention designated CA0071 Z2ZA 01171.

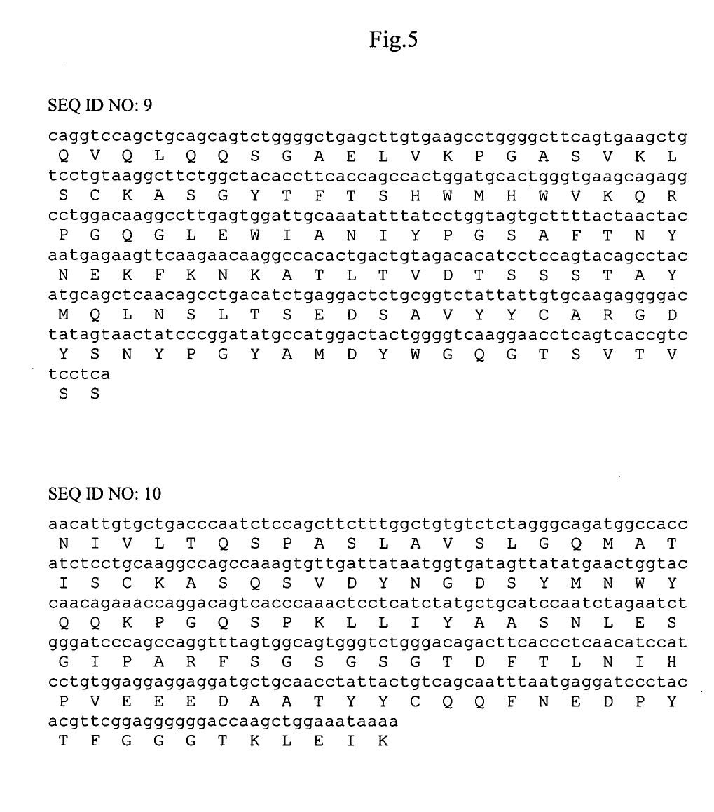

FIG. 6 depicts the heavy chain variable region (SEQ ID NO:11) and light (kappa) chain variable region nucleic acid sequence, together with the translated amino acid sequence, (SEQ ID NO:12) obtained from a preferred antibody of the present invention designated CA0071 Z2ZB 01171.

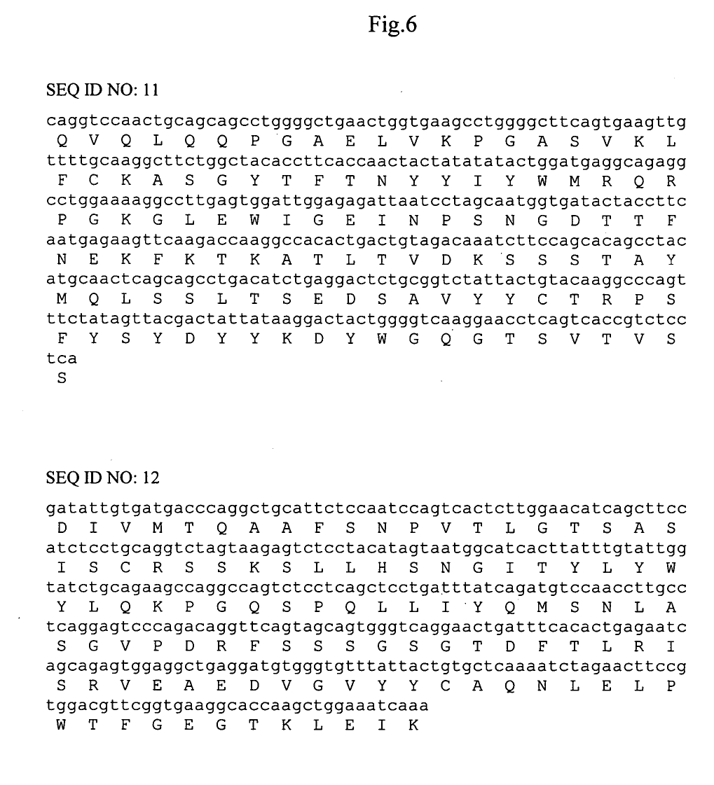

### DETAILED DESCRIPTION OF THE PREFERRED EMBODIMENTS

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. A typical list of nonspecific symptoms in a cardiovasacular disease patient might include one or more of the following: shortness of breath (or dyspnea), chest pain, fever, dizziness, and headache. These symptoms can be common to a number of diseases, the number of which that must be considered by the clinician can be astoundingly broad.

Taking shortness of breath (referred to clinically as “dyspnea”) as an example, this symptom considered in isolation may be indicative of conditions as diverse as asthma, chronic obstructive pulmonary disease (“COPD”), tracheal stenosis, pulmonary injury, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, atrial fibrillation, cardiomyopathy, valvular dysfunction, left ventricle hypertrophy, pericarditis, arrhythmia, pulmonary embolism, metabolic acidosis, chronic bronchitis, pneumonia, anxiety, sepsis, aneurismic dissection, etc. See, e.g., Kelley's Textbook of Internal Medicine, 4^{th} Ed., Lippincott Williams & Wilkins, Philadelphia, Pa., 2000, pp. 2349-2354, “Approach to the Patient With Dyspnea”; Mulrow et al., J. Gen. Int. Med. 8: 383-92 (1993).

Similarly, chest pain, when considered in isolation, may be indicative of stable angina, unstable angina, myocardial ischemia, atrial fibrillation, myocardial infarction, musculoskeletal injury, cholecystitis, gastroesophageal reflux, pulmonary embolism, pericarditis, aortic dissection, pneumonia, anxiety, etc. Moreover, the classification of chest pain as stable or unstable angina (or even mild myocardial infarction) in cases other than definitive myocardial infarction is often completely subjective. The diagnosis, and in this case the distinction, is often made not by angiography, which may quantify the degree of arterial occlusion, but rather by a physician's interpretation of clinical symptoms.

Differential diagnosis refers to methods for diagnosing the particular disease(s) and/or condition(s) underlying the symptoms in a particular subject, based on a comparison of the characteristic features observable from the subject to the characteristic features of those potential diseases. Depending on the breadth of diseases and conditions that must be considered in the differential diagnosis, the types and number of tests that must be ordered by a clinician can be quite large. In the case of dyspnea for example, the clinician may order tests from a group that includes radiography, electrocardiography, exercise treadmill testing, blood chemistry analysis, echocardiography, bronchoprovocation testing, spirometry, pulse oximetry, esophageal pH monitoring, angiography, laryngoscopy, computed tomography, histology, cytology, magnetic resonance imaging, etc. See, e.g., Morgan and Hodge, Am. Fam. Physician 57: 711-16 (1998). The clinician must then integrate information obtained from a battery of tests, leading to a clinical diagnosis that most closely represents the range of symptoms and/or diagnostic test results obtained for the subject.

The present invention describes methods and compositions that can assist the clinician in performing differential diagnosis by assigning a diagnosis or a prognosis to a subject using one or more subject derived markers, one of which is sFLT-1 and/or one or more markers related thereto.

The term “marker” as used herein refers to proteins, polypeptides, phospholipids, small molecules, or other characteristics of one or more subjects to be used as targets for screening test samples obtained from subjects. “Proteins or polypeptides” used as markers in the present invention are contemplated to include any fragments of a particular protein or its biosynthetic parent, in particular, immunologically detectable fragments. “Marker” as used herein may also include derived markers as defined below, and may also include such characteristics as patient's history, age, sex and race, for example.

The term “derived marker” as used herein refers to a value that is a function of one or more measured markers. For example, derived markers may be related to the change over a time interval in one or more measured marker values, may be related to a ratio of measured marker values, may be a marker value at a different measurement time, or may be a complex function such as a panel response function.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆ or NT-proBNP. Because an antibody epitope is on the order of 8 amino acids, an immunoassay will inherently detect such “related markers” so long as the polypeptides contain the epitope(s) necessary to bind to the antibody or antibodies used in the assay. In the foregoing example, if one uses an antibody directed to residues 101-108 of the proBNP molecule in an assay configured to detect BNP, such an assay might also detect proBNP, BNP itself, and any other fragments containing those 8 residues. Thus, an “assay configured to detect” a particular marker may actually measure a population of polypeptides in generating an assay result. Additionally, related markers may be the result of covalent modification of the parent marker, for example by further hydrolysis by proteases, oxidation of methionine residues, ubiquitination, cysteinylation, nitrosylation, glycosylation, etc.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for sFLT-1, and a significant amount (e.g., 50%) of the sFLT-1 that had been originally released into the circulation has now been degraded into smaller fragments. An immunoassay formulated with antibodies that bind a region common to the intact sFLT-1 and the smaller fragment(s) may overestimate the amount of biologically active sFLT-1 present in the sample by 2-fold, potentially resulting in a “false positive” result. Overestimation of certain form(s) present in a sample may also have serious consequences for patient management. Considering the sFLT-1 example again, the sFLT-1 concentration may be used to determine if therapy is effective (e.g., by monitoring sFLT-1 to see if an elevated level is returning to normal upon treatment). The same “false positive” sFLT-1 result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of a protein marker may not detect all of the marker present. In the case of sFLT-1 specifically, this soluble form may bind VEGF or PLGF.

Preferably, the methods described hereinafter utilize one or more markers, including sFLT-1, that are derived from the subject. The term “subject-derived marker” as used herein refers to protein, polypeptide, phospholipid, nucleic acid, prion, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, such as molecules expressed by pathogenic or infectious organisms that are correlated with a particular disease, race, time since onset, sex, etc. Such markers are preferably protein, polypeptide, phospholipid, nucleic acid, prion, or small molecule markers that identify the infectious diseases described above. Exemplary subject derived markers are described herein, and in PCT application no. US03/41453, filed on Dec. 23, 2003, which is hereby incorporated by reference in its entirety.

The term “marker related to myocardial injury” refers to subject-derived markers that are known in the art to be derived from cardiac tissue and that are elevated in the circulation of subjects suffering from damage to the myocardium. Preferred markers of cardiac injury for use in the methods described herein comprise, for example, annexin V, β-enolase, cardiac troponin I (total, free of other troponin polypeptides, and/or complexed with other troponin polypeptides), cardiac troponin T (total, free of other troponin polypeptides, and/or complexed with other troponin polypeptides), creatine kinase-MB, glycogen phosphorylase-BB, heart-type fatty acid binding protein, phosphoglyceric acid mutase-MB, S-100ao, myoglobin, actin, myosin, and lactate dehydrogenase, or markers related thereto. This list is not meant to be limiting.

The term “marker related to apoptosis” refers to subject-derived markers that are elevated in the circulation due to apoptotic processes. Preferred marker(s) related to apoptosis for use in the methods described herein comprise, for example, one or more marker(s) selected from the group consisting of spectrin, cathepsin D, caspase 3, cytochrome c, s-acetyl glutathione, and ubiquitin fusion degradation protein 1 homolog, or markers related thereto. This list is not meant to be limiting.

The term “marker related to blood pressure regulation” refers to subject-derived markers that are known in the art to affect blood pressure regulation. Preferred marker(s) related to blood pressure regulation for use in the methods described herein comprise, for example, one or more marker(s) selected from the group consisting of atrial natriuretic peptide (“ANP”), pro-ANP, B-type natriuretic peptide (“BNP”), NT-pro BNP, pro-BNP C-type natriuretic peptide (“CNP”), pro-CNP, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto. This list is not meant to be limiting.

The term “marker related to inflammation” refers to subject-derived markers that are known in the art to mediate or promote inflammation, activate the complement cascade, and/or stimulate chemotaxis of phagocytes. Preferred marker(s) markers related to inflammation for use in the methods described herein comprise, for example, one or more marker(s) selected from the group consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, e-selectin, glutathione-5-transferase, hypoxia-inducible factor-1α, inducible nitric oxide synthase (“I-NOS”), intracellular adhesion molecule, lactate dehydrogenase, monocyte chemoattractant peptide-1 (“MCP-1”), n-acetyl aspartate, prostaglandin E2, receptor activator of nuclear factor (“RANK”) ligand, lipopolysaccharide binding protein (“LBP”), high mobility group protein-1 (“HMG-1” or “HMGB1”), cystatin C, cell adhesion molecules such as vascular cell adhesion molecule (“VCAM”), intercellular adhesion molecule-1 (“ICAM-1”), intercellular adhesion molecule-2 (“ICAM-2”), and intercellular adhesion molecule-3 (“ICAM-3”), myeloperoxidase (“MPO”), C-reactive protein (“CRP”), interleukins such as IL-1β, IL-6, and IL-8, interleukin-1 receptor agonist, monocyte chemoattractant protein-1, lipocalin-type prostaglandin D synthase, mast cell tryptase, eosinophil cationic protein, haptoglobin, tumor necrosis factor α (“TNF-α”), tumor necrosis factor β, Fas ligand, soluble Fas (Apo-1), TRAIL, TWEAK, fibronectin, macrophage migration inhibitory factor (MIF), and vascular endothelial growth factor (“VEGF”), or markers related thereto. The term “acute phase reactants” as used herein refers to proteins whose concentrations are elevated in response to stressful or inflammatory states that occur during various insults that include infection, injury, surgery, trauma, tissue necrosis, and the like. Acute phase reactant expression and serum concentration elevations are not specific for the type of insult, but rather as a part of the homeostatic response to the insult. This list is not meant to be limiting.

The term “marker related to coagulation and hemostasis” refers to subject-derived markers that are known in the art to be associated with clot presence, or any condition that causes or is a result of fibrinolysis activation. Preferred marker(s) related to coagulation and hemostasis for use in the methods described herein comprise, for example, one or more marker(s) selected from the group consisting of plasmin, fibrinogen, thrombus precursor protein, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, and tissue factor, or markers related thereto. This list is not meant to be limiting.

A table of exemplary markers, and their classification, follows:

| Marker | Classification |
|---|---|
| Myoglobin | Myocardial injury |
| Troponin I and complexes | Myocardial injury |
| Troponin T and complexes | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| ANP | Blood pressure regulation |
| CNP | Blood pressure regulation |
| Kininogen | Blood pressure regulation |
| CGRP II | Blood pressure regulation |
| urotensin II | Blood pressure regulation |
| BNP | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 | Blood pressure regulation |
| angiotensin 2 | Blood pressure regulation |
| angiotensin 3 | Blood pressure regulation |
| Bradykinin | Blood pressure regulation |
| Tachykinin-3 | Blood pressure regulation |
| calcitonin | Blood pressure regulation |
| Endothelin-2 | Blood pressure regulation |
| Endothelin-3 | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C | Coagulation and hemostasis |
| Soluble Endothelial Protein C | Coagulation and hemostasis |
| Receptor (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion | Vascular tissue |
| molecule (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| APRIL (TNF ligand superfamily member 13) | Inflammatory |
| Complement C3a | Inflammatory |
| CCL-5 (RANTES) | Inflammatory |
| CCL-8 (MCP-2) | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-13 (small inducible cytokine B13) | Inflammatory |
| CXCL-16 (small inducible cytokine B16) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| Lipocalin-2 | Inflammatory |
| Lactate dehydrogenase | Inflammatory |
| MCP-1 | Inflammatory |
| MDA-LDL | Inflammatory |
| MMP-1 | Inflammatory |
| MMP-2 | Inflammatory |
| MMP-3 | Inflammatory |
| MMP-9 | Inflammatory |
| TIMP-1 | Inflammatory |
| TIMP-2 | Inflammatory |
| TIMP-3 | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| Transforming growth factor beta | Inflammatory |
| TREM-1 | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| s-acetyl Glutathione | apoptosis |
| cytochrome C | apoptosis |
| Caspase 3 | apoptosis |
| Cathepsin D | apoptosis |
| α-spectrin | apoptosis |

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human animal. Thus, the methods and compositions described herein are applicable to both human and veterinary disease. Further, while a subject is preferably a living animal, the invention described herein may be used in post-mortem analysis as well. Preferred subjects are “patients,” i.e., living humans that are receiving or being evaluated for medical care. This includes persons with no defined illness who are being investigated for signs of pathology.

The phrase “clinical outcome” as used herein refers to the future course of a disease suffered by a subject. Such a clinical outcome may be adverse (e.g., future morbidity or mortality) or may be beneficial (e.g., future improvement in health). In the case of a cardiovascular disease such as ACS for example, an adverse outcome could be a future MI (fatal and/or non-fatal), future stroke (fatal and/or non-fatal), future congestive heart failure, future stable angina, future unstable angina, future need for rehospitalization (that is, the need to readmit a patient for hospital-based treatment following clinical improvement in the patient's present condition sufficient to warrant release from an in-patient setting), future need for coronary revascularization (that is, surgical intervention to improve blood flow to the heart, e.g., by coronary artery bypass grafting, insertion of a stent, percutaneous coronary intervention, etc.), or future death. A clinical outcome is preferably measured within 5 years of the measurement of an sFLT-1 level used to assign a prognosis. A clinical outcome is said to occur within the “near term” if it occurs within about 2 years, preferably within about 12 months, more preferably about 9 months, still more preferably about 6 months, even more preferably about 3 months, yet more preferably within about 1 month, and most preferably within about 7 days of the measurement of an sFLT-1 level used to assign a prognosis.

The term “discrete” as used herein refers to areas of a surface that are non-contiguous. That is, two areas are discrete from one another if a border that is not part of either area completely separates each of the two areas.

The term “independently addressable” as used herein refers to discrete areas of a surface from which an independent signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Preferred antibodies of the present invention comprise at least one polypeptide sequence selected from SEQ ID NOS: 1-6. Preferably, the preferred antibodies comprise (1) a heavy chain variable region comprising the amino acid sequence of SEQ ID NO: 1 and a light chain variable region comprising the amino acid sequence of SEQ ID NO: 2; (2) a heavy chain variable region comprising the amino acid sequence of SEQ ID NO: 3 and a light chain variable region comprising the amino acid sequence of SEQ ID NO: 4; or (3) a heavy chain variable region comprising the amino acid sequence of SEQ ID NO: 5 and a light chain variable region comprising the amino acid sequence of SEQ ID NO: 6. Preferred DNA sequences encoding each of these amino acid sequences are provided as SEQ ID NOS: 7-12, respectively.

The five classes of antibodies are differentiated mainly by their differing heavy chain (IgA, IgD, IgE, IgG and IgM classes have alpha, delta, epsilon, gamma and mu type heavy chains, respectively). There are two classes of light chain; kappa and lambda. In the foregoing preferred antibodies, the light chain sequences described are all of the kappa type, as kappa sequences predominate in the mouse source of these IgG sequences. The preferred light and heavy chain variable regions referred to in the previous paragraph may be used in a variety of methods known in the art to derive additional antibodies that bind to the same or related epitopes to those bound by the preferred antibodies. Such methods may be found, for example, in Antibody Engineering Methods and Protocols, B. Lo, ed., Humana Press, 2004. Such additional antibodies, which may be used in place of or together with the preferred antibodies to provide assays, devices, and kits of the present invention. These additional antibodies may be of any Ig class (e.g. IgG, IgM, IgD, IgE, IgA) or subclass (e.g., IgG1, IgG2, IgG3 or IgG4), may include lambda light chain sequences, may be intact antibodies or may be antigen-binding portions such as are described above, may be single chain antibodies, etc. The antibodies of the present invention may find use as monoclocal populations, or as part of a polyclonal antibody (e.g., formed by pooling two or more individual monoclonals).

An antibody that “binds to a related epitope,” as that term is used herein, means that binding of the new antibody to sFLT-1 is inhibited at least in part by equal concentrations of the original antibody (e.g., one of the preferred antibodies described above), or binding of the original antibody (e.g., one of the preferred antibodies described above) to sFLT-1 is inhibited at least in part by equal concentrations of the new antibody. In one example, chain shuffling may be used to obtain a humanized antibody that binds at or near to one of the preferred antibodies of the present invention. In preferred embodiments, An antibody that “binds to a related epitope” is one that, when present at equal concentrations with one of the preferred antibodies described herein, inhibits binding to sFLT-1 by one of the preferred antibodies described herein, or is inhibited in its binding to sFLT-1 by one of the preferred antibodies described herein, by at least 10%, more preferably at least 25%, still more preferably at least 50%, and most preferably at least 75%. An antibody that “binds to the same epitope” as that term is used herein, means that binding of the new antibody to sFLT-1 is inhibited by at least 90% by equal concentrations of the original antibody, or binding of the original antibody to sFLT-1 is inhibited by at least 90% by equal concentrations of the new antibody.

Use of sFLT-1 as a Prognostic Marker

As described herein, sFLT-1 assay results are predictive of the future clinical course of patients with one or more cardiovascular conditions. Furthermore, the combination of sFLT-1 levels with other markers (e.g., markers related to myocardial injury, markers related to inflammation, markers related to blood pressure regulation, markers related to apoptosis, markers related to coagulation, etc., may improve the predictive value of sFLT-1. Likewise, certain characteristics such as ST-segment depression, age, smoking status, lipid levels, diabetes, ejection fraction, hypertension, and/or prior MI may also be used as additional prognostic indicators that may be combined with an sFLT-1 level in a subject sample.

While described in exemplary embodiments in terms of cardiovascular conditions, sFLT-1 assays may also be applied to prognosis according to the methods described herein in other diseases and conditions in which inflammation is manifested. Such diseases and conditions include Systemic Inflammatory Response Syndrome (“SIRS”), sepsis, severe sepsis, septic shock, infectious diseases, inflammatory bowel disease, pneumonia, nephritis, arthritis, tissue rejection, vasculitis, burns, fractures, pericarditis, myocarditis, endocarditis, Alzheimer's disease, Parkinson's disease, ALS, lupus, pancreatitis, cancer, trauma, etc. This list is not meant to be limiting.

Use of sFLT-1 as a Diagnostic Marker

As also described herein, levels of sFLT-1 may also be used in assigning a diagnosis to patients with cardiovascular disorders. In exemplary embodiments, it is demonstrated that individuals with acute myocardial infarction and angina had increased levels of sFLT-1 in comparison to age-matched normal subjects. This result is contrary to that seen previously using assays for sFAS. See, e.g., Chung et al., Eur. Heart J. 23: 1604-1608, 2002.

As in the case of prognosis, sFLT-1 assays may be applied to the diagnosis of cardiovascular conditions as defined herein. In preferred embodiments, sFLT-1 is preferably applied to diagnosis of an acute coronary syndrome. In various embodiments, the methods are applied to diagnose a subject suffering from non-ST-elevation ACS, ST-elevation ACS, unstable angina, non-ST-elevation non-Q wave MI, ST-elevation non-Q wave MI, and/or transmural (Q-wave) MI. In addition, sFLT-1 levels may also be applied to diagnosis according to the methods described herein in other diseases and conditions in which inflammation is manifested as described herein, including Systemic Inflammatory Response Syndrome (“SIRS”), sepsis, severe sepsis, septic shock, infectious diseases, inflammatory bowel disease, pneumonia, nephritis, arthritis, tissue rejection, vasculitis, burns, fractures, pericarditis, myocarditis, endocarditis, Alzheimer's disease, Parkinson's disease, ALS, lupus, pancreatitis, cancer, and trauma.

Combination of sFLT-1 with Other Markers

In traditional methods to evaluate marker levels in the diagnosis or prognosis of disease, a “threshold” for a marker of interest is typically established, and the concentration of that marker in a sample is compared to that threshold amount; an amount greater than the pre-established threshold is indicative of one state (e.g., disease), and an amount less than the pre-established threshold is indicative of another state (e.g., normal). For example, the American Heart Association has stated that a cardiac troponin I concentration greater that the 99th percentile concentration in the normal population should be used to rule in myocardial infarction.

In certain preferred embodiments, a diagnosis and/or prognosis may be assigned based on the contributions of a plurality of markers. When combining markers, a threshold may be established for each marker of interest. The concentration of each marker in a sample is then compared to its appropriate threshold amount. A particular diagnosis/prognosis may be assigned, depending on the outcome of each comparison. One skilled in the art will recognize that univariate analysis of markers can be performed and the data from the univariate analyses of multiple markers can be combined to form panels of markers to differentiate different disease conditions. In addition, multivariate methods for combining markers are well known to those of skill in the art. See, e.g., Di Fabio et al., Dig. Surg. 21:128-133, 2004; Latini et al., Eur. Heart J. 25(4):292-9, 2004.

In certain embodiments, the present invention may utilize an evaluation of the plurality of markers as a unitary whole. In a simple example, the ratio of two or more markers, rather than an absolute amount of the markers, may be used to determine a diagnosis/prognosis. Even more preferably, however, a particular “fingerprint” pattern of changes in such a panel of markers may, in effect, act as a specific diagnostic or prognostic indicator. Methods for determining a “panel response value” that integrates a plurality of marker concentrations into a single result are described in International Application No. US03/41426, filed Dec. 23, 2003, which is hereby incorporated in its entirety.

In developing a panel of markers, data for a number of potential markers may be obtained from a group of subjects by testing for the presence or level of certain markers. The group of subjects is divided into two sets. The first set includes subjects who have been confirmed as having a disease, outcome, or, more generally, being in a first condition state. For example, this first set of patients may be those diagnosed with acute myocardial infarction that died as a result of that disease. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or diagnosed with acute myocardial infarction that lived to a particular endpoint of interest.

The data obtained from subjects in these sets preferably includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

A single marker often is incapable of definitively identifying a subject as falling within a first or second group in a prospective fashion. For example, if a patient is measured as having a marker level that falls within an overlapping region in the distribution of diseased and non-diseased subjects, the results of the test may be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject 0) is expressed as:
Rⱼ=ΣwᵢIᵢⱼ,
where i is the marker index, j is the subject index, wᵢ is the weighting coefficient for marker i, I is the indicator value to which the marker level for marker i is mapped for subject j, and Σ is the summation over all candidate markers i. This panel response value may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

To allow a determination of test accuracy, a “gold standard” test criterion may be selected which allows selection of subjects into two or more groups for comparison by the foregoing methods. In the case of sepsis, this gold standard may be recovery of organisms from culture of blood, urine, pleural fluid, cerebrospinal fluid, peritoneal fluid, synnovial fluid, sputum, or other tissue specimens. This implies that those negative for the gold standard are free of sepsis; however, as discussed above, 50% or more of patients exhibiting strong clinical evidence of sepsis are negative on culture. In this case, those patients showing clinical evidence of sepsis but a negative gold standard result may be omitted from the comparison groups. Alternatively, an initial comparison of confirmed sepsis subjects may be compared to normal healthy control subjects. In the case of a prognosis, mortality is a common test criterion.

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures:

at least 75% sensitivity, combined with at least 75% specificity;

ROC curve area of at least 0.6, more preferably 0.7, still more preferably at least 0.8, even more preferably at least 0.9, and most preferably at least 0.95; and/or

a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

The diagnostic and/or prognostic panels of the present invention may comprise 2, 3, 4, 5, 6, 7, 8, 9, 10, 15, 20, or more or individual markers. Preferred panels comprise, in addition to sFLT-1, one or more additional markers independently selected from the group consisting of specific markers of cardiac injury, markers related to blood pressure regulation, markers related to inflammation, markers related to coagulation and hemostasis, and markers related to apoptosis. Exemplary markers in each of these groups are described herein. These markers may be combined in various combinations. For example, preferred panels may comprise sFLT-1 and 1, 2, 3, 4, 5, 6, 7, or more of the following markers: BNP, proBNP, NT-proBNP, BNP₃₋₁₀₈, caspase-3, CKMB, C-reactive protein, D-dimer, heart-type fatty acid binding protein, IL-1ra, IL-8, MMP-9, myeloperoxidase, myoglobin, placental growth factor, free cardiac troponin I, free cardiac troponin T, complexed cardiac troponin I, complexed cardiac troponin T, free and complexed cardiac troponin I, free and complexed cardiac troponin T, total cardiac troponin, and thrombus precursor protein, or markers related thereto.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of ACS, identification of the severity of the event, identification of the disease severity, and identification of the patient's outcome, including risk of future events.

A panel consisting of the markers referenced above may be constructed to provide relevant information related to differential diagnosis and/or prognosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses.

In practice, the sensitivity and specificity of a marker for a particular diagnosis or prognosis is typically assessed using a “diseased” population and a “control” (e.g., a normal) population. While the terms “diseased” and “control” are used for convenience herein to refer to these populations, these terms refer to a first subject population exhibiting some characteristic of interest, and a second subject population not exhibiting that characteristic. That characteristic might be the presence or absence of a disease, a risk of some future outcome, etc. Receiver Operating Characteristic curves, or “ROC” curves, may be calculated by plotting the value of a variable versus its relative frequency in the “control” and “disease” populations. For any particular marker, a distribution of marker levels for subjects exhibiting and not exhibiting the characteristic of interest will likely overlap. Such a test need not absolutely distinguish “control” from “disease” with 100% accuracy, and the area of overlap indicates where the test cannot distinguish the control population from the disease population. A threshold value for the test is selected, above which (or below which, depending on how a marker changes with the disease) the test is considered to be indicative of one state or condition in a subject (e.g., disease, outcome, etc.) and below which the test is considered to be indicative of another state or condition in the subject. The area under the ROC curve is a measure of the probability that the perceived measurement will allow correct identification of a characteristic of interest. These methods are well known in the art. See, e.g., Hanley et al., Radiology 143: 29-36 (1982).

Measures of test accuracy may be obtained as described in Fischer et al., Intensive Care Med. 29: 1043-51, 2003; Zhou et al., Statistical Methods in Diagnostic Medicine, John Wiley & Sons, 2002; and Motulsky, Intuitive Biostatistics, Oxford University Press, 1995; and other publications well known to those of skill in the art, and used to determine the effectiveness of a given marker or panel of markers. These measures include sensitivity and specificity, predictive values, likelihood ratios, diagnostic odds ratios, hazard ratios, and ROC curve areas. As discussed above, suitable tests may exhibit one or more of the following results on these various measures:

A ROC curve area of greater than about 0.5, more preferably greater than about 0.7, still more preferably greater than about 0.8, even more preferably greater than about 0.85, and most preferably greater than about 0.9;

a positive or negative likelihood ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less;

an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; and/or

a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less.

Measures of diagnostic accuracy such as those discussed above are often reported together with confidence intervals or p values. These may be calculated by methods well known in the art. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

As is described in detail above, polypeptide markers of interest may be subject to hydrolysis by proteases, oxidation of methionine residues, ubiquitination, cysteinylation, nitrosylation, glycosylation, etc. In addition, sFLT-1 may also be oligomerized. The artisan may consider these modifications when designing measurement strategies. For example, sandwich assays may be designed to recognize only oligomerized forms by selecting a first sandwich-forming antibody that binds to sFLT-1 sequences, and a second antibody that binds only if the sFLT-1 is oligomerized.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting and/or Monitoring a Treatment Regimen

The appropriate treatments for various types of cardiovascular disease may be large and diverse. However, once a diagnosis is obtained, the clinician can readily select a treatment regimen that is compatible with the diagnosis. Accordingly, the present invention provides methods of early differential diagnosis to allow for appropriate intervention in acute time windows. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

Upon treatment, changes in the predicted prognosis of a patient may be monitored by the methods described herein. Any improvement (or lack thereof) may be assessed, and further clinical decisions made based (at least partly) upon this information. The skilled artisan will understand that additional information on health status from other clinical tests (e.g., electrocardiography, exercise treadmill testing, blood chemistry analysis, echocardiography, bronchoprovocation testing, spirometry, pulse oximetry, esophageal pH monitoring, angiography, laryngoscopy, computed tomography, histology, cytology, magnetic resonance imaging) may also be used to supplement the monitoring features of the present invention.

### EXAMPLES

The following examples serve to illustrate the present invention. These examples are in no way intended to limit the scope of the invention.

### Example 1

### Study Population

A detailed description of the design of the AtheroGene study has been outlined previously. See, e.g., Blankenberg et al., Circulation 106:24-30, 2002. Briefly, between November 1996 and June 2000, 1340 patients who underwent coronary angiography at the Department of Medicine II of the Johannes Gutenberg-University Mainz or the Bundeswehrzentralkrankenhaus Koblenz, and who had at least one stenosis >30% diagnosed in a major coronary artery, were enrolled in a cardiovascular registry. Exclusion criteria were evidence of hemodynamically significant valvular heart disease, surgery or trauma within the prior month, known cardiomyopathy, known malignant diseases, febrile conditions, or oral anticoagulant therapy within the prior 4 weeks.

Study participants had German nationality. The study was approved by the local ethics committee. Participation was voluntary and each subject gave written informed consent. The mean age of patients was 61.7±9.9 years, 75% were men, 25% were current smokers, 72% had an history of hypertension and 28% of diabetes mellitus, 47% had had a previous myocardial infarction. At enrolment, 35% of patients were taking statins and 59% beta-blockers.

### Example 2

### Laboratory Methods

Markers were measured using standard immunoassay techniques. These techniques involved the use of antibodies to specifically bind the protein targets.

Typically, a monoclonal antibody directed against a selected marker is biotinylated using N-hydroxysuccinimide biotin (NHS-biotin) at a ratio of about 5 NRS-biotin moieties per antibody. The antibody-biotin conjugate is then added to wells of a standard avidin 384 well microtiter plate, and antibody conjugate not bound to the plate is removed. This forms the “anti-marker” in the microtiter plate. Another monoclonal antibody directed against the same marker is conjugated to alkaline phosphatase using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.). Immunoassays are performed on a TECAN Genesis RSP 200/8 Workstation. Biotinylated antibodies are pipetted into microtiter plate wells previously coated with avidin and incubated for 60 min. The solution containing unbound antibody is removed, and the wells washed with a wash buffer, consisting of 20 mM borate (pH 7.42) containing 150 mM NaCl, 0.1% sodium azide, and 0.02% Tween-20. The plasma samples (10 μL) are pipeted into the microtiter plate wells, and incubated for 60 min. The sample is then removed and the wells washed with a wash buffer. The antibody-alkaline phosphatase conjugate is then added to the wells and incubated for an additional 60 min, after which time, the antibody conjugate is removed and the wells washed with a wash buffer. A substrate, (AttoPhos®, Promega, Madison, Wis.) is added to the wells, and the rate of formation of the fluorescent product was related to the concentration of the marker in the patient samples.

### Example 3

### sFLT-1 as a Diagnostic Marker for Acute Myocardial Infarction

Plasma samples were measured using a commercially available sandwich immunoassay (R&D Systems) that binds for detection sFLT-1 (pg/ml). The same samples were also assayed using immunoassays that bind for detection the following analytes: BNP (pg/mL), BNP₃₋₁₀₈ (pg/mL), caspase-3 (ng/mL), CKMB (ng/mL), C-reactive protein (CRP, μg/mL), D-dimer (DDIM, μg/mL), heart-type fatty acid binding protein (hFABP, ng/mL), IL-1 receptor agonist (IL-1ra, pg/mL), IL-8 (pg/mL), MMP-9 (ng/mL), myeloperoxidase (MPO, ng/mL), myoglobin (MYO, ng/mL), placental growth factor (PLGF, ng/mL), cardiac troponin I (TNI, ng/mL), and thrombus precursor protein (TpP, μg/mL). Univariate ROC areas for each assay in the diagnosis of acute myocardial infarction were determined by comparing the results obtained from a disease group to an age-matched normal population. Confidence intervals were calculated using the SAS software package, version 8.01 (SAS Institute Inc., Cary, N.C., USA).

In addition, panels of markers were formed by combining the sFLT-1 assay results with each of the other analyte assay results in combinations of 2, 3, 4, and 5 markers using the methods described in PCT Application US03/41426, filed Dec. 23, 2003. Briefly, individual threshold concentrations for the markers are not used as cutoffs per se, but are used as values to which the assay values for each patient are compared and normalized. A window factor was used to calculate the minimum and maximum values above and below the cutoff. Assay values above the maximum are set to the maximum and assay values below the minimum are set to the minimum. The absolute values of the weights for the individual markers adds up to 1. A panel response result is calculated using the cutoff, window, and weighting factors for each sample. The panel response results for the entire population of disease and control subjects are subjected to ROC analysis as is commonly performed for individual markers, and a MultiMarker Index™ value is selected to yield the desired sensitivity and specificity for the panel. Because each assay was not performed on each and every individual, the number of disease and control individuals reported below may vary between comparisons.

Average sFLT-1 values for various subject groups were as follows:

| | |
|---|---|
| Age-matched controls: | 32.53 |
| AMI: | 214.95 |
| NSTEMI (TNT−) | 297.22 |
| NSTEMI (TNI+) | 399.54 |
| STEMI (TNI−) | 778.63 |
| STEMI (TNI+) | 753.41 |
| SA (TNI−) | 210.66 |
| UA (TNI−) | 260.94 |

As shown in Table 1, the univariate ROC area obtained using an assay that binds sFLT-1 was superior to the univariate ROC area obtained from all other analyte assays performed. But combination of sFLT-1 with one or more additional marker assays could result in an improved ROC area for the diagnosis of acute myocardial infarction as compared to the univariate use of sFLT-1 assay results:

TABLE 1
Diagnosis of acute myocardial infarction using sFLT-1 alone and in panels

| | Univariate | | | Panel | | | | N | |
|---|---|---|---|---|---|---|---|---|---|
| Markers | ROC Area | 95% Confidence | Interval | ROC Area | Panel ROC SD | 95% Confidence | Interval | # Diseased | # Control |
| FLT | 0.912 | 0.816 | 1.008 | 0.929 | 0 | 0.880 | 0.978 | 325 | 225 |
| BNP 3-108 | 0.665 | 0.569 | 0.761 | | | | | | |
| BNP | 0.899 | 0.809 | 0.989 | 0.972 | 0 | 0.926 | 1.000 | 370 | 257 |
| FLT | 0.896 | 0.806 | 0.989 | | | | | | |
| FLT | 0.913 | 0.820 | 1.006 | 0.93 | 0 | 0.883 | 0.977 | 342 | 244 |
| Caspase-3 | 0.603 | 0.510 | 0.696 | | | | | | |
| CKMB | 0.81 | 0.720 | 0.900 | 0.92 | 0.001 | 0.874 | 0.966 | 370 | 257 |
| FLT | 0.896 | 0.806 | 0.986 | | | | | | |
| FLT | 0.896 | 0.806 | 0.986 | 0.935 | 0.001 | 0.889 | 0.981 | 370 | 257 |
| CRP | 0.736 | 0.646 | 0.826 | | | | | | |
| FLT | 0.896 | 0.806 | 0.986 | 0.919 | 0.001 | 0.873 | 0.965 | 370 | 257 |
| DDIM | 0.638 | 0.548 | 0.728 | | | | | | |
| FLT | 0.907 | 0.811 | 1.003 | 0.915 | 0.002 | 0.866 | 0.964 | 329 | 222 |
| hFABP | 0.689 | 0.593 | 0.785 | | | | | | |
| FLT | 0.913 | 0.817 | 1.009 | 0.912 | 0.003 | 0.863 | 0.961 | 334 | 224 |
| IL-1ra | 0.542 | 0.446 | 0.638 | | | | | | |
| FLT | 0.913 | 0.819 | 1.007 | 0.913 | 0.001 | 0.865 | 0.961 | 337 | 242 |
| IL-8 | 0.532 | 0.438 | 0.626 | | | | | | |
| FLT | 0.912 | 0.818 | 1.006 | 0.912 | 0.002 | 0.865 | 0.959 | 342 | 244 |
| MMP-9 | 0.391 | 0.297 | 0.485 | | | | | | |
| FLT | 0.912 | 0.818 | 1.006 | 0.934 | 0.003 | 0.886 | 0.982 | 344 | 239 |
| MPO | 0.843 | 0.749 | 0.937 | | | | | | |
| FLT | 0.896 | 0.806 | 0.986 | 0.91 | 0.001 | 0.864 | 0.956 | 370 | 257 |
| MYO | 0.688 | 0.598 | 0.778 | | | | | | |
| FLT | 0.896 | 0.805 | 0.987 | 0.896 | 0.002 | 0.850 | 0.942 | 362 | 256 |
| PLGF | 0.505 | 0.414 | 0.596 | | | | | | |
| FLT | 0.896 | 0.806 | 0.986 | 0.968 | 0 | 0.922 | 1.000 | 370 | 257 |
| TNI | 0.901 | 0.811 | 0.991 | | | | | | |
| FLT | 0.911 | 0.818 | 1.004 | 0.95 | 0.001 | 0.902 | 0.998 | 340 | 243 |
| TpP | 0.69 | 0.597 | 0.783 | | | | | | |
| FLT | 0.896 | 0.806 | 0.986 | 0.943 | 0.001 | 0.897 | 0.989 | 370 | 257 |
| DDIM | 0.638 | 0.548 | 0.728 | | | | | | |
| CKMB | 0.81 | 0.720 | 0.900 | | | | | | |
| FLT | 0.896 | 0.806 | 0.986 | 0.987 | 0 | 0.941 | 1.000 | 370 | 257 |
| BNP | 0.899 | 0.809 | 0.989 | | | | | | |
| TNI | 0.901 | 0.811 | 0.991 | | | | | | |
| FLT | 0.896 | 0.806 | 0.986 | 0.968 | 0.001 | 0.922 | 1.000 | 370 | 257 |
| TN | 0.901 | 0.811 | 0.991 | | | | | | |
| CKMB | 0.81 | 0.720 | 0.900 | | | | | | |
| FLT | 0.896 | 0.806 | 0.986 | 0.974 | 0.001 | 0.928 | 1.000 | 370 | 257 |
| TN | 0.901 | 0.811 | 0.991 | | | | | | |
| CRP | 0.736 | 0.646 | 0.826 | | | | | | |
| FLT | 0.896 | 0.806 | 0.986 | 0.973 | 0 | 0.927 | 1.000 | 370 | 257 |
| TN | 0.901 | 0.811 | 0.991 | | | | | | |
| DDIM | 0.638 | 0.548 | 0.728 | | | | | | |
| FLT | 0.912 | 0.818 | 1.006 | 0.977 | 0.004 | 0.929 | 1.000 | 334 | 239 |
| TNI | 0.897 | 0.803 | 0.991 | | | | | | |
| MPO | 0.843 | 0.749 | 0.937 | | | | | | |
| FLT | 0.896 | 0.806 | 0.986 | 0.967 | 0.001 | 0.921 | 1.000 | 370 | 257 |
| TNI | 0.901 | 0.811 | 0.991 | | | | | | |
| MYO | 0.688 | 0.598 | 0.778 | | | | | | |
| FLT | 0.896 | 0.805 | 0.987 | 0.968 | 0 | 0.922 | 1.000 | 362 | 256 |
| TNI | 0.905 | 0.814 | 0.996 | | | | | | |
| PLGF | 0.505 | 0.414 | 0.596 | | | | | | |
| FLT | 0.896 | 0.805 | 0.987 | 0.972 | 0.001 | 0.926 | 1.000 | 362 | 256 |
| BNP | 0.897 | 0.806 | 0.988 | | | | | | |
| PLGF | 0.505 | 0.414 | 0.596 | | | | | | |
| FLT | 0.896 | 0.805 | 0.987 | 0.921 | 0.002 | 0.875 | 0.967 | 362 | 256 |
| CKMB | 0.812 | 0.721 | 0.903 | | | | | | |
| PLGF | 0.505 | 0.414 | 0.596 | | | | | | |
| FLT | 0.896 | 0.805 | 0.987 | 0.935 | 0.001 | 0.889 | 0.981 | 362 | 256 |
| CRP | 0.735 | 0.644 | 0.826 | | | | | | |
| PLGF | 0.505 | 0.414 | 0.596 | | | | | | |
| FLT | 0.896 | 0.805 | 0.987 | 0.918 | 0.001 | 0.872 | 0.964 | 362 | 256 |
| DDIM | 0.638 | 0.547 | 0.729 | | | | | | |
| PLGF | 0.505 | 0.414 | 0.596 | | | | | | |
| FLT | 0.913 | 0.818 | 1.008 | 0.934 | 0.002 | 0.886 | 0.982 | 328 | 238 |
| MPO | 0.842 | 0.747 | 0.937 | | | | | | |
| PLGF | 0.516 | 0.421 | 0.611 | | | | | | |
| FLT | 0.896 | 0.805 | 0.987 | 0.911 | 0.002 | 0.865 | 0.957 | 362 | 256 |
| MYO | 0.692 | 0.601 | 0.783 | | | | | | |
| PLGF | 0.692 | 0.601 | 0.783 | | | | | | |
| FLT | 0.896 | 0.806 | 0.986 | 0.976 | 0.001 | 0.930 | 1.000 | 370 | 257 |
| BNP | 0.899 | 0.809 | 0.989 | | | | | | |
| CKMB | 0.81 | 0.720 | 0.900 | | | | | | |
| FLT | 0.896 | 0.806 | 0.986 | 0.974 | 0.001 | 0.928 | 1.000 | 370 | 257 |
| BNP | 0.899 | 0.809 | 0.989 | | | | | | |
| CRP | 0.736 | 0.646 | 0.826 | | | | | | |
| FLT | 0.896 | 0.806 | 0.986 | 0.973 | 0.001 | 0.927 | 1.000 | 370 | 257 |
| BNP | 0.899 | 0.809 | 0.989 | | | | | | |
| DDIM | 0.638 | 0.548 | 0.728 | | | | | | |
| FLT | 0.896 | 0.806 | 0.986 | 0.973 | 0.001 | 0.927 | 1.000 | 370 | 257 |
| BNP | 0.899 | 0.809 | 0.989 | | | | | | |
| MYO | 0.688 | 0.598 | 0.778 | | | | | | |
| FLT | 0.896 | 0.806 | 0.986 | 0.943 | 0.001 | 0.897 | 0.989 | 370 | 257 |
| DDIM | 0.638 | 0.548 | 0.728 | | | | | | |
| CKMB | 0.81 | 0.720 | 0.900 | | | | | | |
| FLT | 0.896 | 0.806 | 0.986 | 0.941 | 0.002 | 0.895 | 0.987 | 370 | 257 |
| DDIM | 0.638 | 0.548 | 0.728 | | | | | | |
| CRP | 0.736 | 0.646 | 0.826 | | | | | | |
| FLT | 0.912 | 0.818 | 1.006 | 0.941 | 0.002 | 0.893 | 0.989 | 334 | 239 |
| MPO | 0.843 | 0.749 | 0.937 | | | | | | |
| MYO | 0.678 | 0.584 | 0.772 | | | | | | |
| FLT | 0.896 | 0.806 | 0.986 | 0.924 | 0.003 | 0.878 | 0.970 | 370 | 257 |
| MYO | 0.638 | 0.548 | 0.728 | | | | | | |
| DDIM | 0.688 | 0.598 | 0.778 | | | | | | |
| FLT | 0.896 | 0.806 | 0.986 | 0.947 | 0.002 | 0.901 | 0.993 | 370 | 257 |
| CRP | 0.736 | 0.646 | 0.826 | | | | | | |
| CKMB | 0.81 | 0.720 | 0.900 | | | | | | |
| FLT | 0.896 | 0.806 | 0.986 | 0.939 | 0.001 | 0.893 | 0.985 | 370 | 257 |
| CRP | 0.736 | 0.646 | 0.826 | | | | | | |
| MYO | 0.688 | 0.598 | 0.778 | | | | | | |
| FLT | 0.896 | 0.806 | 0.986 | 0.929 | 0.002 | 0.883 | 0.975 | 370 | 257 |
| CKMB | 0.81 | 0.720 | 0.900 | | | | | | |
| MYO | 0.688 | 0.598 | 0.778 | | | | | | |
| FLT | 0.912 | 0.818 | 1.006 | 0.974 | 0.001 | 0.926 | 1.000 | 334 | 239 |
| MPO | 0.843 | 0.749 | 0.937 | | | | | | |
| BNP | 0.9 | 0.806 | 0.994 | | | | | | |
| FLT | 0.912 | 0.818 | 1.006 | 0.953 | 0.003 | 0.905 | 1.000 | 334 | 239 |
| MPO | 0.811 | 0.717 | 0.905 | | | | | | |
| CKMB | 0.843 | 0.749 | 0.937 | | | | | | |
| FLT | 0.912 | 0.818 | 1.006 | 0.951 | 0.002 | 0.903 | 0.999 | 334 | 239 |
| MPO | 0.843 | 0.749 | 0.937 | | | | | | |
| CRP | 0.73 | 0.636 | 0.824 | | | | | | |
| FLT | 0.912 | 0.818 | 1.006 | 0.942 | 0.002 | 0.894 | 0.990 | 334 | 239 |
| MPO | 0.843 | 0.749 | 0.937 | | | | | | |
| DDIM | 0.628 | 0.534 | 0.722 | | | | | | |
| FLT | 0.912 | 0.818 | 1.006 | 0.941 | 0.002 | 0.893 | 0.989 | 334 | 239 |
| MPO | 0.843 | 0.749 | 0.937 | | | | | | |
| MYO | 0.678 | 0.584 | 0.772 | | | | | | |
| FLT | 0.896 | 0.805 | 0.987 | 0.986 | 0.001 | 0.940 | 1.000 | 362 | 256 |
| BNP | 0.897 | 0.806 | 0.988 | | | | | | |
| TNI | 0.905 | 0.814 | 0.996 | | | | | | |
| PLGF | 0.505 | 0.414 | 0.596 | | | | | | |
| FLT | 0.899 | 0.809 | 0.989 | 0.987 | 0.001 | 0.941 | 1.000 | 370 | 257 |
| BNP | 0.896 | 0.806 | 0.986 | | | | | | |
| TNI | 0.901 | 0.811 | 0.991 | | | | | | |
| CKMB | 0.81 | 0.720 | 0.900 | | | | | | |
| FLT | 0.896 | 0.806 | 0.986 | 0.986 | 0.001 | 0.940 | 1.000 | 370 | 257 |
| BNP | 0.899 | 0.809 | 0.989 | | | | | | |
| TNI | 0.901 | 0.811 | 0.991 | | | | | | |
| CRP | 0.736 | 0.646 | 0.826 | | | | | | |
| FLT | 0.896 | 0.806 | 0.986 | 0.987 | 0.001 | 0.941 | 1.000 | 370 | 257 |
| BNP | 0.899 | 0.809 | 0.989 | | | | | | |
| TNI | 0.901 | 0.811 | 0.991 | | | | | | |
| DDIM | 0.638 | 0.548 | 0.728 | | | | | | |
| FLT | 0.912 | 0.818 | 1.006 | 0.99 | 0.001 | 0.942 | 1.000 | 334 | 239 |
| BNP | 0.9 | 0.806 | 0.994 | | | | | | |
| TNI | 0.897 | 0.803 | 0.991 | | | | | | |
| MPO | 0.843 | 0.749 | 0.937 | | | | | | |
| FLT | 0.896 | 0.806 | 0.986 | 0.987 | 0.002 | 0.941 | 1.000 | 370 | 257 |
| BNP | 0.899 | 0.809 | 0.989 | | | | | | |
| TNI | 0.901 | 0.811 | 0.991 | | | | | | |
| MYO | 0.688 | 0.598 | 0.778 | | | | | | |
| FLT | 0.896 | 0.805 | 0.987 | 0.986 | 0.001 | 0.940 | 1.000 | 362 | 256 |
| BNP | 0.897 | 0.806 | 0.988 | | | | | | |
| TN | 0.905 | 0.814 | 0.996 | | | | | | |
| PLGF | 0.505 | 0.414 | 0.596 | | | | | | |
| FLT | 0.896 | 0.805 | 0.987 | 0.986 | 0.002 | 0.940 | 1.000 | 362 | 256 |
| BNP | 0.897 | 0.806 | 0.988 | | | | | | |
| TN | 0.905 | 0.814 | 0.996 | | | | | | |
| CKMB | 0.812 | 0.721 | 0.903 | | | | | | |
| PLGF | 0.505 | 0.414 | 0.596 | | | | | | |
| FLT | 0.896 | 0.805 | 0.987 | 0.986 | 0.001 | 0.940 | 1.000 | 362 | 256 |
| BNP | 0.897 | 0.806 | 0.988 | | | | | | |
| TNI | 0.905 | 0.814 | 0.996 | | | | | | |
| PLGF | 0.505 | 0.414 | 0.596 | | | | | | |
| CRP | 0.735 | 0.644 | 0.826 | | | | | | |
| FLT | 0.896 | 0.805 | 0.987 | 0.987 | 0.001 | 0.941 | 1.000 | 362 | 256 |
| BNP | 0.897 | 0.806 | 0.988 | | | | | | |
| TNI | 0.905 | 0.814 | 0.996 | | | | | | |
| PLGF | 0.505 | 0.414 | 0.596 | | | | | | |
| DDIM | 0.638 | 0.547 | 0.729 | | | | | | |
| FLT | 0.913 | 0.818 | 1.008 | 0.989 | 0.001 | 0.941 | 1.000 | 328 | 238 |
| BNP | 0.898 | 0.803 | 0.993 | | | | | | |
| TNI | 0.901 | 0.806 | 0.996 | | | | | | |
| PLGF | 0.516 | 0.421 | 0.611 | | | | | | |
| MPO | 0.842 | 0.747 | 0.937 | | | | | | |
| FLT | 0.896 | 0.805 | 0.987 | 0.986 | 0 | 0.940 | 1.000 | 362 | 256 |
| BNP | 0.897 | 0.806 | 0.988 | | | | | | |
| TNI | 0.905 | 0.814 | 0.996 | | | | | | |
| PLGF | 0.505 | 0.414 | 0.596 | | | | | | |
| MYO | 0.692 | 0.601 | 0.783 | | | | | | |

### Example 4

### sFLT-1 as a Diagnostic Marker for Acute Coronary Syndrome

Using the MultiMarker Index™ value obtained by comparing acute myocardial infarction to age matched controls, the ability of each panel to diagnose acute coronary syndrome was also analyzed. Acute coronary syndrome (including acute myocardial infarction, unstable angina, and stable angina) was also subdivided in various analyses into acute myocardial infarction (AMI) 0-3 hours post event, acute myocardial infarction 0-6 hours post event, non-ST elevation myocardial infarction (NSTEMI), non-ST elevation myocardial infarction 0-3 hours post event, non-ST elevation myocardial infarction 0-6 hours post event, ST elevation myocardial infarction (STEMI), ST elevation myocardial infarction 0-3 hours post event, ST elevation myocardial infarction 0-6 hours post event, unstable angina (UA), stable angina (SA), and troponin I-negative (TNI−) non-ST elevation myocardial infarction, ST elevation myocardial infarction, unstable angina, and stable angina:

TABLE 2

| | Panel # | | | |
|---|---|---|---|---|
| | 1 | 2 | 3 | 4 |
| Marker(s) in panel | BNP₃₋₁₀₈ | BNP₃₋₁₀₈ | BNP₃₋₁₀₈ | BNP₃₋₁₀₈ |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 225 | 225 | 225 | 225 |
| Disease n | 13 | 38 | 6 | 14 |
| Ave ROC Area | 0.99 | 0.98 | 1.00 | 0.97 |
| | Panel # | | | |
| | 5 | 6 | 7 | 8 |
| Marker(s) in panel | BNP₃₋₁₀₈ | BNP₃₋₁₀₈ | BNP₃₋₁₀₈ | BNP₃₋₁₀₈ |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 225 | 225 | 225 | 225 |
| Disease n | 221 | 7 | 24 | 96 |
| Ave ROC Area | 0.92 | 0.99 | 0.98 | 0.95 |
| | Panel # | | | |
| | 9 | 10 | 11 | 12 |
| Marker(s) in panel | BNP₃₋₁₀₈ | BNP₃₋₁₀₈ | BNP₃₋₁₀₈ | BNP₃₋₁₀₈ |
| with sFLT-1 | | | | |
| Comparison | UA 0-3 h v AMN | UA 0-6 h v AMN | UA all times v | SA all times v |
| | | | AMN | AMN |
| Normal n | 225 | 225 | 225 | 225 |
| Disease n | 6 | 13 | 319 | 875 |
| Ave ROC Area | 1.00 | 0.96 | 0.84 | 0.77 |
| | Panel # | | | |
| | 13 | 14 | 15 | 16 |
| Marker(s) in panel | BNP | BNP | BNP | BNP |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 16 | 45 | 8 | 17 |
| Ave ROC Area | 0.98 | 0.99 | 1.00 | 1.00 |
| | Panel # | | | |
| | 17 | 18 | 19 | 20 |
| Marker(s) in panel | BNP | BNP | BNP | BNP |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 259 | 8 | 28 | 102 |
| Ave ROC Area | 0.97 | 0.97 | 0.98 | 0.99 |
| | Panel # | | | |
| | 21 | 22 | 23 | 24 |
| Marker(s) in panel | BNP | BNP | BNP | BNP |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 7 | 14 | 345 | 1022 |
| Ave ROC Area | 0.99 | 0.97 | 0.90 | 0.83 |
| | Panel # | | | |
| | 25 | 26 | 27 | 28 |
| Marker(s) in panel | Caspase-3 | Caspase-3 | Caspase-3 | Caspase-3 |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 244 | 244 | 244 | 244 |
| Disease n | 14 | 40 | 6 | 14 |
| Ave ROC Area | 0.96 | 0.98 | 1.00 | 0.97 |
| | Panel # | | | |
| | 29 | 30 | 31 | 32 |
| Marker(s) in panel | Caspase-3 | Caspase-3 | Caspase-3 | Caspase-3 |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 244 | 244 | 244 | 244 |
| Disease n | 234 | 8 | 26 | 99 |
| Ave ROC Area | 0.92 | 0.94 | 0.98 | 0.95 |
| | Panel # | | | |
| | 33 | 34 | 35 | 36 |
| Marker(s) in panel | Caspase-3 | Caspase-3 | Caspase-3 | Caspase-3 |
| with sFLT-1 | | | | |
| Comparison | UA 0-3 h v AMN | UA 0-6 h v AMN | UA all times v | SA all times v |
| | | | AMN | AMN |
| Normal n | 244 | 244 | 244 | 244 |
| Disease n | 7 | 14 | 333 | 928 |
| Ave ROC Area | 1.00 | 0.96 | 0.85 | 0.77 |
| | Panel # | | | |
| | 37 | 38 | 39 | 40 |
| Marker(s) in panel | CKMB | CKMB | CKMB | CKMB |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 16 | 45 | 8 | 17 |
| Ave ROC Area | 0.98 | 0.97 | 1.00 | 0.96 |
| | Panel # | | | |
| | 41 | 42 | 43 | 44 |
| Marker(s) in panel | CKMB | CKMB | CKMB | CKMB |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 259 | 8 | 28 | 102 |
| Ave ROC Area | 0.90 | 0.95 | 0.98 | 0.96 |
| | Panel # | | | |
| | 45 | 46 | 47 | 48 |
| Marker(s) in panel | CKMB | CKMB | CKMB | CKMB |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 7 | 14 | 345 | 1022 |
| Ave ROC Area | 1.00 | 0.94 | 0.78 | 0.68 |
| | Panel # | | | |
| | 49 | 50 | 51 | 52 |
| Marker(s) in panel | CRP | CRP | CRP | CRP |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 16 | 45 | 8 | 17 |
| Ave ROC Area | 0.97 | 0.97 | 1.00 | 0.95 |
| | Panel # | | | |
| | 53 | 54 | 55 | 56 |
| Marker(s) in panel | CRP | CRP | CRP | CRP |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 259 | 8 | 28 | 102 |
| Ave ROC Area | 0.92 | 0.95 | 0.98 | 0.97 |
| | Panel # | | | |
| | 57 | 58 | 59 | 60 |
| Marker(s) in panel | CRP | CRP | CRP | CRP |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 7 | 14 | 345 | 1022 |
| Ave ROC Area | 1.00 | 0.93 | 0.82 | 0.72 |
| | Panel # | | | |
| | 61 | 62 | 63 | 64 |
| Marker(s) in panel | DDIM | DDIM | DDIM | DDIM |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 16 | 45 | 8 | 17 |
| Ave ROC Area | 0.98 | 0.96 | 1.00 | 0.93 |
| | Panel # | | | |
| | 65 | 66 | 67 | 68 |
| Marker(s) in panel | DDIM | DDIM | DDIM | DDIM |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 259 | 8 | 28 | 102 |
| Ave ROC Area | 0.90 | 0.95 | 0.98 | 0.96 |
| | Panel # | | | |
| | 69 | 70 | 71 | 72 |
| Marker(s) in panel | DDIM | DDIM | DDIM | DDIM |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 7 | 14 | 345 | 1022 |
| Ave ROC Area | 1.00 | 0.98 | 0.84 | 0.74 |
| | Panel # | | | |
| | 73 | 74 | 75 | 76 |
| Marker(s) in panel | hFABP | hFABP | hFABP | hFABP |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 222 | 222 | 222 | 222 |
| Disease n | 12 | 36 | 5 | 12 |
| Ave ROC Area | 0.97 | 0.99 | 1.00 | 0.99 |
| | Panel # | | | |
| | 77 | 78 | 79 | 80 |
| Marker(s) in panel | hFABP | hFABP | hFABP | hFABP |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 222 | 222 | 222 | 222 |
| Disease n | 224 | 7 | 24 | 96 |
| Ave ROC Area | 0.90 | 0.96 | 0.99 | 0.96 |
| | Panel # | | | |
| | 81 | 82 | 83 | 84 |
| Marker(s) in panel | hFABP | hFABP | hFABP | hFABP |
| with sFLT-1 | | | | |
| Comparison | UA 0-3 h v AMN | UA 0-6 h v AMN | UA all times v | SA all times v |
| | | | AMN | AMN |
| Normal n | 222 | 222 | 222 | 222 |
| Disease n | 6 | 12 | 320 | 865 |
| Ave ROC Area | 1.00 | 0.98 | 0.82 | 0.74 |
| | Panel # | | | |
| | 85 | 86 | 87 | 88 |
| Marker(s) in panel | IL-1ra | IL-1ra | IL-1ra | IL-1ra |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 224 | 224 | 224 | 224 |
| Disease n | 14 | 40 | 6 | 14 |
| Ave ROC Area | 0.98 | 0.98 | 1.00 | 0.96 |
| | Panel # | | | |
| | 89 | 90 | 91 | 92 |
| Marker(s) in panel | IL-1ra | IL-1ra | IL-1ra | IL-1ra |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 224 | 224 | 224 | 224 |
| Disease n | 229 | 8 | 26 | 96 |
| Ave ROC Area | 0.90 | 0.97 | 0.99 | 0.95 |
| | Panel # | | | |
| | 93 | 94 | 95 | 96 |
| Marker(s) in panel | IL-1ra | IL-1ra | IL-1ra | IL-1ra |
| with sFLT-1 | | | | |
| Comparison | UA 0-3 h v AMN | UA 0-6 h v AMN | UA all times v | SA all times v |
| | | | AMN | AMN |
| Normal n | 224 | 224 | 224 | 224 |
| Disease n | 7 | 14 | 323 | 902 |
| Ave ROC Area | 1.00 | 0.938 | 0.82 | 0.74 |
| | Panel # | | | |
| | 97 | 98 | 99 | 100 |
| Marker(s) in panel | IL-8 | IL-8 | IL-8 | IL-8 |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 242 | 242 | 242 | 242 |
| Disease n | 12 | 36 | 5 | 12 |
| Ave ROC Area | 0.98 | 0.96 | 1.00 | 0.93 |
| | Panel # | | | |
| | 101 | 102 | 103 | 104 |
| Marker(s) in panel | IL-8 | IL-8 | IL-8 | IL-8 |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 242 | 242 | 242 | 242 |
| Disease n | 224 | 7 | 24 | 96 |
| Ave ROC Area | 0.90 | 0.96 | 0.99 | 0.95 |
| | Panel # | | | |
| | 105 | 106 | 107 | 108 |
| Marker(s) in panel | IL-8 | IL-8 | IL-8 | IL-8 |
| with sFLT-1 | | | | |
| Comparison | UA 0-3 h v AMN | UA 0-6 h v AMN | UA all times v | SA all times v |
| | | | AMN | AMN |
| Normal n | 242 | 242 | 242 | 242 |
| Disease n | 6 | 12 | 320 | 865 |
| Ave ROC Area | 1.00 | 0.93 | 0.81 | 0.73 |
| | Panel # | | | |
| | 109 | 110 | 111 | 112 |
| Marker(s) in panel | MMP-9 | MMP-9 | MMP-9 | MMP-9 |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 244 | 244 | 244 | 244 |
| Disease n | 14 | 40 | 6 | 14 |
| Ave ROC Area | 0.99 | 0.97 | 1.00 | 0.94 |
| | Panel # | | | |
| | 113 | 114 | 115 | 116 |
| Marker(s) in panel | MMP-9 | MMP-9 | MMP-9 | MMP-9 |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 244 | 244 | 244 | 244 |
| Disease n | 234 | 8 | 26 | 99 |
| Ave ROC Area | 0.90 | 0.97 | 0.99 | 0.95 |
| | Panel # | | | |
| | 117 | 118 | 119 | 120 |
| Marker(s) in panel | MMP-9 | MMP-9 | MMP-9 | MMP-9 |
| with sFLT-1 | | | | |
| Comparison | UA 0-3 h v AMN | UA 0-6 h v AMN | UA all times v | SA all times v |
| | | | AMN | AMN |
| Normal n | 244 | 244 | 244 | 244 |
| Disease n | 7 | 14 | 334 | 931 |
| Ave ROC Area | 1.00 | 0.91 | 0.82 | 0.74 |
| | Panel # | | | |
| | 121 | 122 | 123 | 124 |
| Marker(s) in panel | MPO | MPO | MPO | MPO |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 239 | 239 | 239 | 239 |
| Disease n | 14 | 40 | 6 | 14 |
| Ave ROC Area | 0.96 | 0.98 | 1.00 | 0.98 |
| | Panel # | | | |
| | 125 | 126 | 127 | 128 |
| Marker(s) in panel | MPO | MPO | MPO | MPO |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 239 | 239 | 239 | 239 |
| Disease n | 229 | 8 | 26 | 96 |
| Ave ROC Area | 0.0.93 | 0.93 | 0.98 | 0.96 |
| | Panel # | | | |
| | 129 | 130 | 131 | 132 |
| Marker(s) in panel | MPO | MPO | MPO | MPO |
| with sFLT-1 | | | | |
| Comparison | UA 0-3 h v AMN | UA 0-6 h v AMN | UA all times v | SA all times v |
| | | | AMN | AMN |
| Normal n | 239 | 239 | 239 | 239 |
| Disease n | 7 | 14 | 322 | 912 |
| Ave ROC Area | 1.00 | 0.91 | 0.85 | 0.76 |
| | Panel # | | | |
| | 133 | 134 | 135 | 136 |
| Marker(s) in panel | MYO | MYO | MYO | MYO |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 12 | 36 | 5 | 12 |
| Ave ROC Area | 0.97 | 0.98 | 1.00 | 0.98 |
| | Panel # | | | |
| | 137 | 138 | 139 | 140 |
| Marker(s) in panel | MYO | MYO | MYO | MYO |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 224 | 7 | 24 | 96 |
| Ave ROC Area | 0.89 | 0.95 | 0.98 | 0.95 |
| | Panel # | | | |
| | 141 | 142 | 143 | 144 |
| Marker(s) in panel | MYO | MYO | MYO | MYO |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 6 | 12 | 320 | 865 |
| Ave ROC Area | 1.00 | 0.0.97 | 0.81 | 0.71 |
| | Panel # | | | |
| | 145 | 146 | 147 | 148 |
| Marker(s) in panel | PLGF | PLGF | PLGF | PLGF |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 256 | 256 | 256 | 256 |
| Disease n | 16 | 44 | 8 | 16 |
| Ave ROC Area | 0.98 | 96 | 1.00 | 0.90 |
| | Panel # | | | |
| | 149 | 150 | 151 | 152 |
| Marker(s) in panel | PLGF | PLGF | PLGF | PLGF |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 256 | 256 | 256 | 256 |
| Disease n | 253 | 8 | 28 | 100 |
| Ave ROC Area | 0.88 | 0.97 | 0.99 | 0.95 |
| | Panel # | | | |
| | 153 | 154 | 155 | 156 |
| Marker(s) in panel | PLGF | PLGF | PLGF | PLGF |
| with sFLT-1 | | | | |
| Comparison | UA 0-3 h v AMN | UA 0-6 h v AMN | UA all times v | SA all times v |
| | | | AMN | AMN |
| Normal n | 256 | 256 | 256 | 256 |
| Disease n | 7 | 14 | 343 | 1011 |
| Ave ROC Area | 1.00 | 0.92 | 0.81 | 0.72 |
| | Panel # | | | |
| | 157 | 158 | 159 | 160 |
| Marker(s) in panel | TNI | TNI | TNI | TNI |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 257 | 257 | 257 | 257 |
| Disease n | 16 | 45 | 8 | 17 |
| Ave ROC Area | 1.00 | 1.00 | 1.00 | 0.99 |
| | Panel # | | | |
| | 161 | 162 | 163 | 164 |
| Marker(s) in panel | TNI | TNI | TNI | TNI |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 257 | 257 | 257 | 257 |
| Disease n | 259 | 8 | 28 | 102 |
| Ave ROC Area | 0.96 | 1.00 | 1.00 | 1.00 |
| | Panel # | | | |
| | 165 | 166 | 167 | 168 |
| Marker(s) in panel | TNI | TNI | TNI | TNI |
| with sFLT-1 | | | | |
| Comparison | UA 0-3 h v AMN | UA 0-6 h v AMN | UA all times v | SA all times v |
| | | | AMN | AMN |
| Normal n | 257 | 257 | 257 | 257 |
| Disease n | 7 | 14 | 345 | 1022 |
| Ave ROC Area | 1.00 | 0.99 | 0.82 | 0.72 |
| | Panel # | | | |
| | 169 | 170 | 171 | 172 |
| Marker(s) in panel | TpP | TpP | TpP | TpP |
| with sFLT-1 | | | | |
| Comparison | AMI 0-3 h v | AMI 0-6 h v | NSTEMI 0-3 h v | NSTEMI 0-6 h v |
| | AMN | AMN | AMN | AMN |
| Normal n | 243 | 243 | 243 | 243 |
| Disease n | 13 | 39 | 6 | 14 |
| Ave ROC Area | 0.93 | 0.97 | 1.00 | 0.98 |
| | Panel # | | | |
| | 173 | 174 | 175 | 176 |
| Marker(s) in panel | TpP | TpP | TpP | TpP |
| with sFLT-1 | | | | |
| Comparison | NSTEMI all | STEMI 0-3 h v | STEMI 0-6 h v | STEMI all times |
| | times v AMN | AMN | AMN | v AMN |
| Normal n | 243 | 243 | 243 | 243 |
| Disease n | 233 | 7 | 25 | 98 |
| Ave ROC Area | 0.95 | 0.86 | 0.96 | 0.96 |
| | Panel # | | | |
| | 177 | 178 | 179 | 180 |
| Marker(s) in panel | TpP | TpP | TpP | TpP |
| with sFLT-1 | | | | |
| Comparison | UA 0-3 h v AMN | UA 0-6 h v AMN | UA all times v | SA all times v |
| | | | AMN | AMN |
| Normal n | 243 | 243 | 243 | 243 |
| Disease n | 7 | 14 | 333 | 917 |
| Ave ROC Area | 1.00 | 0.98 | 0.90 | 0.86 |
| | Panel # | | | |
| | 181 | 182 | 183 | 184 |
| Marker(s) in panel | TNI, BNP, MPO, | TNI, BNP, MPO, | TNI, BNP, MPO, | TNI, BNP, MPO, |
| with sFLT-1 | MYO, CKMB | MYO, CKMB | MYO, CKMB | MYO, CKMB |
| Comparison | AMI 0-3 h v | AMI 0-6 h v | NSTEMI 0-3 h v | NSTEMI 0-6 h v |
| | AMN | AMN | AMN | AMN |
| Normal n | 239 | 239 | 239 | 239 |
| Disease n | 14 | 40 | 6 | 14 |
| Ave ROC Area | 0.96 | 0.98 | 0.98 | 0.99 |
| | Panel # | | | |
| | 185 | 186 | 187 | 188 |
| Marker(s) in panel | TNI, BNP, MPO, | TNI, BNP, MPO, | TNI, BNP, MPO, | TNI, BNP, MPO, |
| with sFLT-1 | MYO, CKMB | MYO, CKMB | MYO, CKMB | MYO, CKMB |
| Comparison | NSTEMI all | STEMI 0-3 h v | STEMI 0-6 h v | STEMI all times |
| | times v AMN | AMN | AMN | v AMN |
| Normal n | 239 | 239 | 239 | 239 |
| Disease n | 229 | 8 | 26 | 96 |
| Ave ROC Area | 0.96 | 0.95 | 0.98 | 0.99 |
| | Panel # | | | |
| | 189 | 190 | 191 | 192 |
| Marker(s) in panel | TNI, BNP, MPO, | TNI, BNP, MPO, | TNI, BNP, MPO, | TNI, BNP, MPO, |
| with sFLT-1 | MYO, CKMB | MYO, CKMB | MYO, CKMB | MYO, CKMB |
| Comparison | NSTEMI TNI− v | NSTEMI NTI+ v | STEMI TNI− v | STEMI TNI+ v |
| | AMN | AMN | AMN | AMN |
| Normal n | 239 | 239 | 239 | 239 |
| Disease n | 62 | 167 | 19 | 77 |
| Ave ROC Area | 0.87 | 1.00 | 0.94 | 1.00 |

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of diagnosing a cardiovascular condition in a subject or assigning a prognostic risk of one or more future clinical outcomes to a subject suffering from a cardiovascular condition, the method comprising:
performing an assay configured to detect soluble FLT-1 on a sample obtained from said subject to generate an assay result; and
performing one or more assays configured to detect one or more markers selected from the group consisting of BNP, prbBNP, NT-proBNP, BNP₃₋₁₀₈, caspase-3, CKMB, C-reactive protein, D-dimer, heart-type fatty acid binding protein, IL-1ra, IL-8, MMP-9, myeloperoxidase, myoglobin, placental growth factor, free cardiac troponin I, free cardiac troponin T, complexed cardiac troponin I, complexed cardiac troponin T, free and complexed cardiac troponin I, free and complexed cardiac troponin T, total cardiac troponin, and thrombus precursor protein, on the same sample or one or more different samples obtained from said subject to generate one or more additional assay results; and
relating the assay results to the presence or absence of the cardiovascular condition in the subject, or to the prognostic risk of one or more clinical outcomes for the subject.

2. A method according to claim 1, wherein said one or more clinical outcomes are selected from the group consisting of death, stroke, myocardial infarction, rehospitalization, coronary revascularization, and congestive heart failure.

3. A method according to claim 1, wherein said cardiovascular condition is selected from the group consisting of acute coronary syndrome, atherosclerosis, ischemic stroke, intracerebral hemorrhage, subarachnoid hemorrhage, transient ischemic attack, systolic dysfunction, diastolic dysfunction, aneurysm, aortic dissection, myocardial ischemia, angina pectoris, stable angina, unstable angina, acute myocardial infarction, acute ST elevation myocardial infarction, acute non-ST elevation myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, and peripheral vascular disease.

4. A method according to claim 1, wherein said cardiovascular condition is an acute coronary syndrome.

5. A method according to claim 1, wherein said cardiovascular condition is selected from the group consisting of acute myocardial infarction, acute ST elevation myocardial infarction, acute non-ST elevation myocardial infarction, acute troponin negative myocardial infarction, acute troponin negative ST elevation myocardial infarction, acute troponin negative non-ST elevation myocardial infarction, stable angina, and unstable angina.

6. A method according to claim 1, wherein the method further comprises performing one or more assays configured to detect one or more additional markers other than those listed in claim 1, on the same sample or one or more different samples obtained from said subject to generate one or more additional assay results for use in said correlating step.

7. A method according to claim 1, wherein said method provides a ROC area of at least 0.75 for the diagnosis of myocardial infarction or for the prognostic risk of mortality.

8. A method according to claim 1, wherein said method provides a ROC area of at least 0.9 for the diagnosis of myocardial infarction or for the prognostic risk of mortality.

9. A method according to claim 1, wherein said method provides an odds ratio of about 4 or greater or about 0.25 or less for the diagnosis of myocardial infarction or for the prognostic risk of mortality.

10. A method according to claim 1, wherein said method provides a hazard ratio of about 1.25 or greater or about 0.8 or less for the diagnosis of myocardial infarction or for the prognostic risk of mortality.

11. A method according to claim 1, wherein the method comprises performing one or more assays configured to detect one or more of BNP, proBNP, NT-proBNP, or BNP₃₋₁₀₈.

12. A method according to claim 1, wherein the method comprises performing one or more assays configured to detect one or more of free cardiac troponin I, complexed cardiac troponin I, free and complexed cardiac troponin I, free cardiac troponin T, complexed cardiac troponin T, or free and complexed cardiac troponin T.

13. A method according to claim 1, wherein the method comprises performing an assay configured to detect C-reactive protein.

14. A method according to claim 1, wherein the method comprises performing an assay configured to detect myeloperoxidase.

15. A method according to claim 1, wherein the method comprises performing an assay configured to detect CKMB.

16. A method according to claim 1, wherein the method comprises performing an assay configured to detect D-dimer.

17. A method according to claim 1, wherein the method comprises performing an assay configured to detect MMP-9.

18. A method according to claim 1, wherein the method comprises performing an assay configured to detect heart-type fatty acid binding protein.

19. A method according to claim 1, wherein the method comprises performing assays configured to detect at least two markers selected from the group consisting of BNP, NT-proBNP, CKMB, D-dimer, heart-type fatty acid binding protein, MMP-9, myeloperoxidase, myoglobin, placental growth factor, free and complexed cardiac troponin I, and free and complexed cardiac troponin T.

20. A method according to claim 1, wherein the method comprises performing assays configured to detect at least three markers selected from the group consisting of BNP, NT-proBNP, CKMB, D-dimer, heart-type fatty acid binding protein, MMP-9, myeloperoxidase, myoglobin, placental growth factor, free and complexed cardiac troponin I, and free and complexed cardiac troponin T.

21. A method according to claim 1, wherein the method comprises performing assays configured to detect at least four markers selected from the group consisting of BNP, NT-proBNP, CKMB, D-dimer, heart-type fatty acid binding protein, MMP-9, myeloperoxidase, myoglobin, placental growth factor, free and complexed cardiac troponin I, and free and complexed cardiac troponin T.

22. A method according to claim 1, wherein the method comprises performing assays configured to detect at least five markers selected from the group consisting of BNP, NT-proBNP, CKMB, D-dimer, heart-type fatty acid binding protein, MMP-9, myeloperoxidase, myoglobin, placental growth factor, free and complexed cardiac troponin I, and free and complexed cardiac troponin T.

23. A method according to claim 1, wherein the method comprises performing assays configured to detect at least six markers selected from the group consisting of BNP, NT-proBNP, CKMB, D-dimer, heart-type fatty acid binding protein, MMP-9, myeloperoxidase, myoglobin, placental growth factor, free and complexed cardiac troponin I, and free and complexed cardiac troponin T.

24. A method according to claim 1, wherein the sample is from a human.

25. A method according to claim 1, wherein the sample is selected from the group consisting of blood, serum, and plasma.

26. A method according to claim 1, wherein the assays are immunoassays.

27. A device for performing the method of claim 26, comprising a plurality of discrete locations on a solid phase, each comprising antibodies for performing said assays.

28. A method according to claim 1, wherein the relating step comprises comparing the soluble FLT-1 assay result from the subject to a threshold soluble FLT-1 level, and performing one or more of the following determinations:
diagnosing the presence of a cardiovascular condition if the assay result is greater than the threshold soluble FLT-1 level; or
diagnosing the absence of a cardiovascular condition if the assay result is less than the threshold soluble FLT-1 level; or
assigning an increased likelihood of a poor prognostic outcome if the assay result is greater than the threshold soluble FLT-1 level, relative to a prognostic risk assigned if the assay result is less than the threshold soluble FLT-1 level; or
assigning a decreased likelihood of a poor prognostic outcome if the assay result is less than the threshold soluble FLT-1 level, relative to a prognostic risk assigned if the assay result is greater than the threshold soluble FLT-1 level.

29. A method of diagnosing an acute coronary syndrome in a subject or assigning a prognostic risk of one or more future clinical outcomes to a subject suffering from an acute coronary syndrome, the method comprising:
performing an assay configured to detect soluble FLT-1 on a sample obtained from said subject to generate an assay result; and
relating the assay result to the presence or absence of the cardiovascular condition in the subject, or to the prognostic risk of one or more clinical outcomes for the subject, wherein the relating step comprises comparing the soluble FLT-1 assay result from the subject to a threshold soluble FLT-1 level, and performing one or more of the following determinations:
diagnosing the presence of an acute coronary syndrome if the assay result is greater than the threshold soluble FLT-1 level; or
diagnosing the absence of an acute coronary syndrome if the assay result is less than the threshold soluble FLT-1 level; or
assigning an increased likelihood of a poor prognostic outcome if the assay result is greater than the threshold soluble FLT-1 level, relative to a prognostic risk assigned if the assay result is less than the threshold soluble FLT-1 level; or
assigning a decreased likelihood of a poor prognostic outcome if the assay result is less than the threshold soluble FLT-1 level, relative to a prognostic risk assigned if the assay result is greater than the threshold soluble FLT-1 level.

30. A method according to claim 29, wherein said acute coronary syndrome is a myocardial infarction.

31. A method according to claim 29, wherein said cardiovascular condition is selected from the group consisting of acute myocardial infarction, acute ST elevation myocardial infarction, acute non-ST elevation myocardial infarction, acute troponin negative myocardial infarction, acute troponin negative ST elevation myocardial infarction, acute troponin negative non-ST elevation myocardial infarction, stable angina, and unstable angina.

32. A method according to claim 29, wherein said cardiovascular condition is selected from the group consisting of acute myocardial infarction, acute ST elevation myocardial infarction, acute non-ST elevation myocardial infarction, acute troponin negative myocardial infarction, acute troponin negative ST elevation myocardial infarction, and acute troponin negative non-ST elevation myocardial infarction.

33. A method according to claim 29, wherein the method further comprises performing one or more assays configured to detect one or more additional markers other than those listed in claim 1, on the same sample or one or more different samples obtained from said subject to generate one or more additional assay results for use in said relating step.

34. A method of diagnosing a cardiovascular condition in a subject or assigning a prognostic risk of one or more future clinical outcomes to a subject suffering from a cardiovascular condition, the method comprising:
performing an immunoassay on a sample obtained from said subject, wherein the immunoassay comprises contacting said sample with an assay antibody selected from the group consisting of an antibody comprising a heavy chain variable region comprising the sequence of SEQ ID NO: 1 and a light chain variable region comprising the sequence of SEQ ID NO: 2, an antibody comprising a heavy chain variable region comprising the sequence of SEQ ID NO: 3 and a light chain variable region comprising the sequence of SEQ ID NO: 4, and an antibody comprising a heavy chain variable region comprising the sequence of SEQ ID NO: 5 and a light chain variable region comprising the sequence of SEQ ID NO: 6, or with an antibody that binds to the same epitope or a related epitope as said assay antibody, and generating a signal indicative of the presence or amount of material bound thereby; and
relating the signal generated in said immunoassay to the presence or absence of the cardiovascular condition in the subject, or to the prognostic risk of one or more clinical outcomes for the subject.

35. A method according to claim 34, wherein said cardiovascular condition is an acute coronary syndrome.

36. A method according to claim 34, wherein said cardiovascular condition is selected from the group consisting of acute myocardial infarction, acute ST elevation myocardial infarction, acute non-ST elevation myocardial infarction, acute troponin negative myocardial infarction, acute troponin negative ST elevation myocardial infarction, acute troponin negative non-ST elevation myocardial infarction, stable angina, and unstable angina.

37. A method according to claim 34, wherein said cardiovascular condition is selected from the group consisting of acute myocardial infarction, acute ST elevation myocardial infarction, acute non-ST elevation myocardial infarction, acute troponin negative myocardial infarction, acute troponin negative ST elevation myocardial infarction, and acute troponin negative non-ST elevation myocardial infarction.

38. A method according to claim 34, wherein the method further comprises performing one or more assays configured to detect one or more additional markers other than those listed in claim 1, on the same sample or one or more different samples obtained from said subject to generate one or more additional assay results for use in said correlating step.

39. A method according to claim 34, wherein the relating step comprises determining a concentration of sFLT-1 in said sample using the signal generated in said immunoassay, comparing said concentration to a threshold sFLT-1 concentration, and performing one or more of the following determinations:
diagnosing the presence of a cardiovascular condition if the assay result is greater than the threshold soluble FLT-1 level; or
diagnosing the absence of a cardiovascular condition if the assay result is less than the threshold soluble FLT-1 level; or
assigning an increased likelihood of a poor prognostic outcome if the assay result is greater than the threshold soluble FLT-1 level, relative to a prognostic risk assigned if the assay result is less than the threshold soluble FLT-1 level; or
assigning a decreased likelihood of a poor prognostic outcome if the assay result is less than the threshold soluble FLT-1 level, relative to a prognostic risk assigned if the assay result is greater than the threshold soluble FLT-1 level.

40. A method according to claim 34, wherein the immunoassay is a sandwich immunoassay that utilizes at least two assay antibodies selected from the group consisting of an antibody comprising a heavy chain variable region comprising the sequence of SEQ ID NO: 1 and a light chain variable region comprising the sequence of SEQ ID NO: 2, an antibody comprising a heavy chain variable region comprising the sequence of SEQ ID NO: 3 and a light chain variable region comprising the sequence of SEQ ID NO: 4, and an antibody comprising a heavy chain variable region comprising the sequence of SEQ ID NO: 5 and a light chain variable region comprising the sequence of SEQ ID NO: 6, wherein each assay antibody is optionally replaced by an antibody that binds to the same epitope or a related epitope as said assay antibody.

41. A kit for measuring sFLT-1 in a sample, comprising:
one or more assay antibodies selected from the group consisting of an antibody comprising a heavy chain variable region comprising the sequence of SEQ ID NO: 1 and a light chain variable region comprising the sequence of SEQ ID NO: 2, an antibody comprising a heavy chain variable region comprising the sequence of SEQ ID NO: 3 and a light chain variable region comprising the sequence of SEQ ID NO: 4, and an antibody comprising a heavy chain variable region comprising the sequence of SEQ ID NO: 5 and a light chain variable region comprising the sequence of SEQ ID NO: 6, wherein each assay antibody is optionally replaced by an antibody that binds to the same epitope or a related epitope as said assay antibody.

42. A kit according to claim 41, comprising at least two assay antibodies selected from the group consisting of an antibody comprising a heavy chain variable region comprising the sequence of SEQ ID NO: 1 and a light chain variable region comprising the sequence of SEQ ID NO: 2, an antibody comprising a heavy chain variable region comprising the sequence of SEQ ID NO: 3 and a light chain variable region comprising the sequence of SEQ ID NO: 4, and an antibody comprising a heavy chain variable region comprising the sequence of SEQ ID NO: 5 and a light chain variable region comprising the sequence of SEQ ID NO: 6, wherein each assay antibody is optionally replaced by an antibody that binds to the same epitope or a related epitope as said assay antibody.

43. A kit according to claim 41, further comprising a threshold value to be used for comparison of a measured sFLT-1 value from said sample to a prognosis or diagnosis.

44. A kit according to claim 43, further comprising instructions for use of said threshold value, wherein said instructions comprise one or more of the following:
an instruction to diagnose the presence of an acute coronary syndrome if the concentration of sFLT-1 in said sample is greater than the threshold soluble FLT-1 level; or
an instruction to diagnose the absence of an acute coronary syndrome if the concentration of sFLT-1 in said sample is less than the threshold soluble FLT-1 level; or
an instruction to assign an increased likelihood of a poor prognostic outcome if the concentration of sFLT-1 in said sample is greater than the threshold soluble FLT-1 level, relative to a prognostic risk assigned if the concentration of sFLT-1 in said sample is less than the threshold soluble FLT-1 level; or
an instruction to assign a decreased likelihood of a poor prognostic outcome if the concentration of sFLT-1 in said sample is less than the threshold soluble FLT-1 level, relative to a prognostic risk assigned if the concentration of sFLT-1 in said sample is greater than the threshold soluble FLT-1 level.


SEQUENCE LISTING (12 sequences)

SEQ ID NO: 1 - protein, 120 aa, Mus musculus
EVQLQQSGPELVKPGASVKMSCKASGYTFPDYNMHWVKQKPGQGLEWIGYINPYNDGTEYNEKFKGKATLTSDKSSSTAYMDLSSLTSEDSAVYYCGRGWGDYWYFDVWGAGTTVTVSSA

SEQ ID NO: 2 - protein, 107 aa, Mus musculus
DIVMTQSQKFMSTSVGDRVSVTCKASQNVGTNVAWYQQKPGQFPKTLIYSASYRYSGVPDRFTGSGSGTDFTLTITNVQSEDLAEYFCQQYNSNPLTFGAGTKLELK

SEQ ID NO: 3 - protein, 122 aa, Mus musculus
QVQLQQSGAELVKPGASVKLSCKASGYTFTSHWMHWVKQRPGQGLEWIANIYPGSAFTNYNEKFKNKATLTVDTSSSTAYMQLNSLTSEDSAVYYCARGDYSNYPGYAMDYWGQGTSVTVSS

SEQ ID NO: 4 - protein, 111 aa, Mus musculus
NIVLTQSPASLAVSLGQMATISCKASQSVDYNGDSYMNWYQQKPGQSPKLLIYAASNLESGIPARFSGSGSGTDFTLNIHPVEEEDAATYYCQQFNEDPYTFGGGTKLEIK

SEQ ID NO: 5 - protein, 121 aa, Mus musculus
QVQLQQPGAELVKPGASVKLFCKASGYTFTNYYIYWMRQRPGKGLEWIGEINPSNGDTTFNEKFKTKATLTVDKSSSTAYMQLSSLTSEDSAVYYCTRPSFYSYDYYKDYWGQGTSVTVSS

SEQ ID NO: 6 - protein, 112 aa, Mus musculus
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGEGTKLEIK

SEQ ID NO: 7 - DNA, 360 bp, Mus musculus
  CDS (1)..(360)
gaggtccagcttcagcagtcaggacctgagctggtaaagcctggggcttcagtgaagatgtcctgcaaggcttctggatacacattccctgactacaacatgcactgggtgaagcagaagcctgggcagggccttgagtggattgggtatattaatccttacaatgatggtactgagtacaatgagaagttcaaaggcaaggccacactgacttcagacaaatcctccagcacagcctacatggatctcagcagcctgacctctgaggactctgcggtctattactgtggaaggggatggggtgactactggtacttcgatgtctggggcgcagggaccacggtcaccgtctcctcagcc

SEQ ID NO: 8 - DNA, 321 bp, Mus musculus
  CDS (1)..(321)
gacattgtgatgacccagtctcaaaaattcatgtccacatcagtaggagacagggtcagcgtcacctgcaaggccagtcagaatgtgggtactaatgtagcctggtatcaacagaaaccagggcaatttcctaaaacactgatttactcggcatcctaccggtacagtggagtccctgatcgcttcacaggcagtggatctgggacagatttcactctcaccatcaccaatgtgcagtctgaagacttggcagagtatttctgtcagcaatataacagcaatcctctcacgttcggtgcggggaccaagctggagctgaaa

SEQ ID NO: 9 - DNA, 366 bp, Mus musculus
  CDS (1)..(366)
caggtccagctgcagcagtctggggctgagcttgtgaagcctggggcttcagtgaagctgtcctgtaaggcttctggctacaccttcaccagccactggatgcactgggtgaagcagaggcctggacaaggccttgagtggattgcaaatatttatcctggtagtgcttttactaactacaatgagaagttcaagaacaaggccacactgactgtagacacatcctccagtacagcctacatgcagctcaacagcctgacatctgaggactctgcggtctattattgtgcaagaggggactatagtaactatcccggatatgccatggactactggggtcaaggaacctcagtcaccgtctcctca

SEQ ID NO: 10 - DNA, 333 bp, Mus musculus
  CDS (1)..(333)
aacattgtgctgacccaatctccagcttctttggctgtgtctctagggcagatggccaccatctcctgcaaggccagccaaagtgttgattataatggtgatagttatatgaactggtaccaacagaaaccaggacagtcacccaaactcctcatctatgctgcatccaatctagaatctgggatcccagccaggtttagtggcagtgggtctgggacagacttcaccctcaacatccatcctgtggaggaggaggatgctgcaacctattactgtcagcaatttaatgaggatccctacacgttcggaggggggaccaagctggaaataaaa

SEQ ID NO: 11 - DNA, 363 bp, Mus musculus
  CDS (1)..(363)
caggtccaactgcagcagcctggggctgaactggtgaagcctggggcttcagtgaagttgttttgcaaggcttctggctacaccttcaccaactactatatatactggatgaggcagaggcctggaaaaggccttgagtggattggagagattaatcctagcaatggtgatactaccttcaatgagaagttcaagaccaaggccacactgactgtagacaaatcttccagcacagcctacatgcaactcagcagcctgacatctgaggactctgcggtctattactgtacaaggcccagtttctatagttacgactattataaggactactggggtcaaggaacctcagtcaccgtctcctca

SEQ ID NO: 12 - DNA, 336 bp, Mus musculus
  CDS (1)..(336)
gatattgtgatgacccaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtggacgttcggtgaaggcaccaagctggaaatcaaa